(19) United States
(12) Patent Application Publication
(10) Pub. No.: US 2021/0259842 A1
(43) Pub. Date: Aug. 26, 2021

(54) BONE IMPLANT DEVICE

(71) Applicant: VERSITECH LIMITED, Hong Kong (CN)

(72) Inventors: Xiaoreng FENG, Hong Kong (CN); Ka-Li Frankie LEUNG, Hong Kong (CN); Christian Xinshuo FANG, Hong Kong (CN)

(73) Assignee: VERSITECH LIMITED, Hong Kong (CN)

(21) Appl. No.: 17/252,055

(22) PCT Filed: Jun. 13, 2019
(86) PCT No.: PCT/CN2019/091056

§ 371 Date: Dec. 14, 2020

(30) Foreign Application Priority Data
Jun. 14, 2018 (US) 62685042

(51) Int. Cl. A61F 2/30; A61B 17/70; A61B 17/86; A61B 17/04

(52) U.S. Cl. CPC A61F 2/30771; A61B 17/7001; A61F 2002/30869; A61B 17/0401; A61B 2017/044; A61B 17/8625

(57) ABSTRACT

An implant device (A1) for engagement with a bone of a patient, the implant device (A1) comprising a distal end (B1), a proximal end (C1), a central shaft (D1) extending therebetween and a longitudinal central axis (E1); the implant device (A1) further including a helical thread portion (F1) extending circumferentially about the central shaft (D1) and extending from the distal end (B1) towards the proximal end (C1) thereof, and a root (G1) at the base of the helical thread portion (F1) adjacent the central shaft (D1), the helical thread portion (F1) including a leading edge (H1) and a trailing edge (I1) both extending at least radially outwardly from the central shaft (D1) and defining the thread portion (F1) therebetween, with the root (G1) of the thread portion (F1) defined therebetween in a direction of the longitudinal central axis (E1) of the implant device (A1); wherein the leading edge (H1) faces in a direction of at least towards the distal end (B1) of the implant device (A1), and the trailing edge (I1) faces at least in a direction of towards the proximal end (C1) of the implant device (A1); and wherein a portion of the trailing edge (I1) extends in a direction towards the proximal end (C1) of the implant (A1) further than the most proximal portion of the root (G1) of the thread portion (F1) such that the portion of the trailing edge (I1) forms a recess (J1) between the central shaft (D1) and the trailing edge (I1).

### TECHNICAL FIELD

The present invention relates to a bone implant device for engagement with bone. More particularly, the present invention provides a bone implant device for reducing loosening thereof in bone material.

### BACKGROUND OF THE INVENTION

Bone implant devices for fixation and engagement with typically include a threaded engagement portion for engagement with and fixation within bone material. Such bone implant devices have numerous applications in the field of orthopaedics, such as when used alone to reduce a fracture or secure fractured bone, to secure and fix other fracture or trauma hardware, such as fracture plate, secure implants such as protheses in the field of arthroplasty.

Other bone implant devices which include a threaded engagement portion for engagement with and fixation in bone include devices such as pedicle screws and suture anchors.

Within the art of bone implant devices and fastener and fixation type devices such as those recited above, which typically include a threaded portion for engagement with bone tissue, there exist numerous problems associated with the biomechanical and biological properties of bone and physiological response of bone in response to the presence of such devices and the loading thereto, which may potentially reduce the integrity of engagement and fixation in bone, and securement of such devices.

By way of example, bone fasteners such as bone screws, nails, and plates may have the effect of weakening or compromising the integrity of surrounding tissue through a physiological mechanism known as stress shielding, which results from bone adjacent a fixation element or implant resorbing due to the absence of localised loading.

Such localised changes in bone tissue adjacent a fixation element, fastener or implant can further result in compromise of a mechanical engagement device, by a further mechanism known termed aseptic loosening, whereby the fit and engagement between orthopedic implants and bone tissue is compromised resulting in a device loosening over time. This may further precipitate loosening and catastrophic failure of the mechanical system, which may be exacerbated by the device crushing and compacting adjacent bone tissue.

Further problems which result include what is known as progressive “cut out”, whereby a device may progressively penetrate through the bone from relative movement between the device and bone, until the device breaks through the cortex entirely.

Such biomechanical problems associated with such devices are often related to, and exacerbated by, biological changes to the processes of bone generation and remodeling.

A common biological change is the loss of bone mass and structural strength due to imbalance in the bone remodeling process, a condition known as osteopenia, or its more extreme form, the progression to osteoporosis.

As global life expectancies of people have risen during the 21st century, an increasing number of otherwise healthy and able elderly people suffer d from painful and debilitating fractures due to osteoporosis. Fractures of the hip, shoulder and spine of a subject are especially prevalent due to the relatively high content of cancellous, or “spongy,” tissue within the larger, load-bearing bones.

In individuals suffering from osteoporosis, these bones often develop numerous cavities and cysts within the spongy bone tissue, that can compromise structural strength and lead to higher fracture and rates.

A common form of treatment of subjects for such fractures is surgical fixation via the implantation of metal rods or screws that secure bone fragments in their original anatomical positions during the healing process.

All bone tissue, in particularly bone tissue already weakened by conditions such as osteoporosis, degenerative disorders, compromised bone stock, are susceptible to complications due to the migration and loosening of devices including implants, fixation devices and bone anchors.

Such migration of the device within bone can cause instability of fracture sites, aseptic loosening, increased stresses on implants and fixation devices, which may precipitate fatigue and failure and upon bone anchors which may cause instability and potential loosening and pull-out, and other complications that reduce overall musculoskeletal health and integrity of bone tissue and bone stability.

As mentioned above, the presence of a device within bone stock may contribute to or cause weakness of the bone through mechanisms such as bone resorption due to stress shielding.

The majority of prior attempts to create implant screws and fasteners and other fixation devices with an improved ability to remain stationary within bone tissue have focused on the use of rigid mechanisms that firmly anchor implants to the surrounding bone tissue. Examples of such mechanisms include expanding metal sleeves, articulated arms, and telescoping fingers designed to penetrate into and grab hold of bone tissue.

### OBJECT OF THE INVENTION

It is an object of the present invention to provide an implant device which overcome or at least partly ameliorate at least some deficiencies as associated with the prior art.

### SUMMARY OF THE INVENTION

In a first aspect, the present invention provides an implant device for engagement with a bone of a patient, said implant device comprising a distal end, a proximal end, a central shaft extending therebetween and a longitudinal central axis; said implant device further including a helical thread portion extending circumferentially about said central shaft and extending from the distal end towards the proximal end thereof, and a root at the base of the helical thread portion adjacent the central shaft, said helical thread portion including a leading edge and a trailing edge both extending at least radially outwardly from the central shaft and defining the thread portion therebetween, with the root of the thread portion defined therebetween in a direction of the longitudinal central axis of the implant device; wherein said leading edge faces in a direction of at least towards the distal end of the implant device, and said trailing edge portion faces at least in a direction of towards the proximal end of the implant device; and wherein a portion of the trailing edge extends in a direction towards the proximal end of the implant further than the most proximal portion of root of the thread such that said portion of the trailing edge forms a recess between the central shaft and the trailing edge.

The portion of said trailing edge defining said recess provides for abutment and engagement with bone tissue of a subject disposed within said recess.

The thread portion may further include a crest portion at the crest of the thread portion. The thread portion extends in at least a direction of from the distal end towards the proximal end, and wherein said crest portion forms a radially outward portion of the thread portion. The crest portion provides an engagement surface for abutment and engagement with bone of a subject radially disposed from said thread portion.

The engagement surface of said crest portion, upon engagement with radially disposed bone adjacent the thread portion, provides for distribution of stress induced in said bone adjacent the crest portion along said engagement surface, and said engagement surface provides for reducing stress concentration in bone adjacent said crest portion.

The crest portion preferably has a greater longitudinal length than that of the root portion in the direction of the longitudinal central axis of the implant device.

The longitudinal length of the thread portion from the most distal portion of the most proximal portion of the thread portion may be greater than the length of the root of the thread portion.

The leading edge of the thread portion may include a first facet for abutment and engagement with bone tissue of a subject, and wherein the trailing edge of thread portion includes a second facet for abutment and engagement with bone tissue of a subject, and wherein said crest portion is disposed between the first facet and the second facet.

In an embodiment of the present invention, the first facet has a substantially planar surface and extends substantially radially outwardly from the distal side of the root portion at the central shaft and extends towards the crest portion.

The second facet may extend from the proximal side of the root portion at the central shaft and extends towards the crest portion.

In another embodiment of the present invention, the second facet is substantially planar and extends from the proximal side of the root portion at the central shaft and extends towards the crest portion at an inclination to the central shaft.

In a further embodiment of the present invention, the trailing edge further includes a third facet, wherein the second and third facets have a substantially planar surface, and wherein the second facet and extends from the proximal side of the root portion at the central shaft and extends towards the third facet, and the third facet extends towards the crest portion.

The trailing edge may further include a third facet, wherein the second and third facets have a substantially planar surface, and wherein the second facet extends substantially radially outwardly from the proximal side of the root portion at the central shaft and extends towards the third facet, and the third facet extends in an inclined direction of from the second facet radially outwardly and proximally towards the crest portion.

In yet another embodiment of the present invention, the trailing edge further includes a third and a fourth facet, wherein the second and third and fourth facets have a substantially planar surface, and wherein the second facet extends substantially radially outwardly from the proximal side of the root portion at the central shaft and extends towards the third facet, and wherein the third facet extends in an direction substantially parallel to the shaft portion from the second facet and towards the fourth facet, and wherein the fourth facet extends from the third facet substantially radially outwardly from the third facet and towards the crest portion.

The engagement surface of the crest portion may be substantially planar and parallel to the longitudinal axis.

Alternatively, the engagement surface of the crest portion may be a curved surface.

The engagement portion of the crest portion may be at least partially provided by the leading edge.

The engagement portion of the crest portion may be at least partially provided by the trailing edge.

The recess is sized and shaped so as to reduce stress concentration induced in bone in respect of bone engaged with and adjacent the tread portion.

The recess is sized and shaped such that upon the implant device and adjacent bone in which the device is embedded being urged towards each other on a first side of the implant, at least a portion of the trailing edge of the thread portion is urged against bone disposed within the recesses on the opposed side of the implant device.

The thread portion may have a constant cross-sectional area and geometry, or alternatively have a varying cross-sectional area and geometry.

The thread portion may have as a constant thread pitch, or may have a varying a constant thread pitch. Preferably, the implant device is formed from a metal or metal alloy material. The metal or metal alloy material may be selected from the group including stainless steel, titanium, titanium alloy, cobalt-chromium alloy or the like.

Alternatively, the implant device may be formed from a polymeric material or polymer based material. The polymeric material or polymer based material may be polyether ether ketone (PEEK).

The implant device is a bone screw. The implant may be an orthopaedic locking screw.

Alternatively, the implant device may be a pedicle screw device, the femoral head engagement element of a dynamic hip screw, bone suture anchor, or an orthopaedic implant prosthesis device.

In a second aspect, the present invention provides a kit comprising one or more implant devices according to the first aspect.

The one or more implant devices may be a bone screw. The kit may further comprise one or more fracture fixation devices.

In a third aspect, the present invention provides a system for fixing a first portion of bone relative to a second portion of bone, said system having 2 or more implant devices according to the first aspect and a bridging member, wherein a first implant device is engageable with the first portion of bone and a second implant device is engageable with the second portion of bone, wherein the distal ends of the implant devices are engageable with said portions of bone and the proximal ends are engageable with said bridging member.

The one or more implant devices may be pedicle screws and the bridging member is a rod, and the system may be a spinal fusion system.

The rod is adjustable so as to provide adjustable movement of the first portion of bone and the second portion of bone relative to each other.

Alternatively, the system may be a trauma fixation system.

In a fourth aspect, the present invention provides an implant device for engagement with a bone of a subject, said implant device comprising a distal end, a proximal end, a central shaft extending therebetween and a longitudinal central axis;
said implant device further including a helical thread portion extending circumferentially about said central shaft and extending from the distal end towards the proximal end thereof, and a root at the base of the helical thread portion adjacent the central shaft, said helical thread portion including:
a leading edge and a trailing edge both extending at least radially outwardly from the central shaft and defining the thread portion therebetween, with the root of the thread portion defined therebetween in a direction of the longitudinal central axis of the implant device, and wherein said leading edge faces in a direction of at least towards the distal end of the implant device, and said trailing edge faces at least in a direction of towards the proximal end of the implant device;
a crest portion at the crest of the thread portion, wherein the thread portion extends in at least a direction of from the distal end towards the proximal end and provides a recess between the central shaft and the thread portion for abutment and engagement with bone adjacent the thread portion, and wherein said crest portion forms a radially outward portion of the thread portion and includes an engagement surface for abutment and engagement with bone of a subject radially disposed from said thread portion

the engagement surface of said crest portion, upon engagement with radially disposed bone adjacent the thread portion, provides for distribution of stress induced in said bone adjacent the crest portion along said engagement surface, and said engagement surface provides for reducing stress concentration in bone adjacent said crest portion.

The recess is sized and shaped such that upon the implant device and adjacent bone in which the device is embedded being urged towards each other on a first side of the implant, at least a portion of the trailing edge of the thread portion is urged against bone disposed within the recesses on the opposed side of the implant device

The trailing edge forms a recess between the central shaft and the trailing edge.

The implant device may be a bone screw. The implant device may be an orthopaedic locking screw.

Alternatively, the implant device is a pedicle screw device, the femoral head engagement element of a dynamic hip screw, bone suture anchor or an orthopaedic implant prosthesis device.

In a fifth aspect, the present invention provides a kit comprising one or more implant devices according to the fifth aspect.

The one or more implant devices may be a bone screw.

The kit may comprise one or more fracture fixation devices.

In a sixth aspect, the present invention provides a system for fixing a first portion of bone relative to a second portion of bone, said system having 2 or more implant devices according to the fourth aspect and a bridging member, wherein a first implant device is engageable with the first portion of bone and a second implant device is engageable with the second portion of bone, wherein the distal ends of the implant devices are engageable with said portions of bone and the proximal ends are engageable with said bridging member.

The one or more implant devices are pedicle screws and the bridging member may be a rod, and the system is a spinal fusion system.

The rod may be adjustable so as to provide adjustable movement of the first portion of bone and the second portion of bone relative to each other.

The system may be a trauma fixation system.

### BRIEF DESCRIPTION OF THE DRAWINGS

In order that a more precise understanding of the above-recited invention can be obtained, a more particular description of the invention briefly described above will be rendered by reference to specific embodiments thereof that are illustrated in the appended drawings. The drawings presented herein may not be drawn to scale and any reference to dimensions in the drawings or the following description is specific to the embodiments disclosed.

FIG. 1 shows a side view of a representation of a bone screw of the Prior Art;

FIG. 2 shows a perspective view of the bone screw of FIG. 1;

FIG. 3 shows a side view of the bone screw of the Prior Art of FIG. 1 and FIG. 2 engaged with a further member shown in section;

FIG. 4 shows a partial sectional view of a portion of the bone screw of the Prior Art of FIGS. 1 to 3;

FIG. 5 shows a perspective schematic view of the bone screw of the Prior Art of FIGS. 1 to 3 engaged with a portion of bone material and a further member;

FIG. 6 illustrates a generic scalar number line measuring stress applied to a small portion of bone tissue;

FIG. 7 shows a perspective sectional view of FIG. 5;

FIG. 8 shows a schematic representation of FIG. 7 with a load applied thereto;

FIG. 9 shows a side view of FIG. 7;

FIG. 10 shows a schematic representation of FIG. 9 with a load applied thereto;

FIG. 11 shows an enlarged sectional view of a portion of FIG. 10;

FIG. 12 shows a sectional side view graphical representation of the bone screw of the Prior Art of FIGS. 1 to 11, for evaluation within a three-dimensional finite element analysis (FEA) model for assessment of load transfer characteristics to adjacent bone material;

FIG. 13 illustrates the range of Von Mises stress from three-dimensional finite element analysis (FEA) of FIG. 12;

FIG. 14 is a graphical representation of the Von Mises stresses induced in bone material adjacent the bone screw of FIG. 12 from three-dimensional finite element analysis (FEA);

FIG. 15 illustrates the range of vertical principal stress from three-dimensional finite element analysis (FEA) of FIG. 12;

FIG. 16 is a graphical representation of the vertical principal stress induced in bone material adjacent the bone screw of FIG. 12 from three-dimensional finite element analysis (FEA);

FIG. 17 illustrates the range of horizontal principal stress from three-dimensional finite element analysis (FEA) of FIG. 12;

FIG. 18 is a graphical representation of the horizontal principal stress induced in bone material adjacent the bone screw of FIG. 12 from three-dimensional finite element analysis (FEA);

FIG. 19A shows a sectional schematic side view of a portion of an implant device according to the present invention, illustrating the principles and features of the present invention;

FIG. 19B shows a further sectional schematic side view of a portion of an implant device according to the present invention, further illustrating the principles and further features of the present invention;

FIG. 20 shows a side view of an embodiment of an implant device according to the present invention;

FIG. 21 shows a perspective view of the implant device of FIG. 20.

FIG. 22 shows a side view of the implant device of FIGS. 20 and 21 engaged with a further member;

FIG. 23 shows an enlarged sectional side view of the implant device of FIGS. 20 to 22;

FIG. 24 shows a perspective side view of the implant device of FIGS. 20 to 22 engaged with a further member and engaged with bone tissue;

FIG. 25 shows a sectional side view of FIG. 24;

FIG. 26 illustrates a generic scalar number line measuring stress applied to a small portion of bone tissue;

FIG. 27 shows a sectional perspective side view of FIG. 25 with as load applied to the bone tissue;

FIG. 28 shows a sectional side view of FIG. 24;

FIG. 29 shows a sectional side view of FIG. 24 with a load applied to the bone tissue;

FIG. 30 shows a portion of an enlarged view of the implant device embodiment of the present invention of FIG. 27;

FIG. 31 shows a detail focused portion of the implant device embodiment of the present invention as in FIG. 29;

FIG. 32 shows an enlarged a sectional; view of the ort implant device embodiment of the present invention as in FIG. 29;

FIG. 33 shows a range of possible values of the dimensions described in FIG. 32;

FIG. 34 shows a range of possible values of the ratios between the dimensions described in FIG. 23;

FIG. 35 illustrates the initial conditions, prior to loading, of a three-dimensional finite element analysis (FEA) model constructed in the mechanical simulation;

FIG. 36 shows the scale of Von Mises stress from the simulation of FIG. 35;

FIG. 37 illustrates the conditions following loading of the model shown in FIG. 35, showing Von Mises stress using the scale in FIG. 36

FIG. 38 shows the scale of vertical principal stress from the simulation of FIG. 35;

FIG. 39 illustrates the conditions following loading of the model shown in FIG. 35, showing vertical principal stress using the scale of FIG. 38.

FIG. 40 shows the scale of horizontal principal stress from the simulation of FIG. 35;

FIG. 41 illustrates the conditions following loading of the model shown in FIG. 35, showing horizontal principal stress using the scale of FIG. 40;

FIG. 42 shows an embodiment of an orthopaedic implant device according to the present invention;

FIG. 43 is an enlarged sectional view of a portion of the embodiment of FIG. 42;

FIG. 44 shows a further embodiment of an orthopaedic implant device according to the present invention;

FIG. 45 is an enlarged sectional view of a portion of the embodiment of FIG. 44;

FIG. 46 shows another embodiment of an orthopaedic implant device according to the present invention;

FIG. 47 is an enlarged sectional view of a portion of the embodiment of FIG. 46;

FIG. 48 shows yet a further embodiment of an orthopaedic implant device according to the present invention;

FIG. 49 is an enlarged sectional view of a portion of the embodiment of FIG. 42;

FIG. 50 shows yet another embodiment of an orthopaedic implant device according to the present invention;

FIG. 51 is an enlarged sectional view of a portion of the embodiment of FIG. 50;

FIG. 52 shows still yet a further embodiment of an orthopaedic implant device according to the present invention;

FIG. 53 is an enlarged sectional view of a portion of the embodiment of FIG. 52;

FIG. 54 shows still yet another embodiment of an orthopaedic implant device according to the present invention;

FIG. 55 is an enlarged sectional view of a portion of the embodiment of FIG. 54;

FIG. 56 shows an alternate embodiment of an orthopaedic implant device according to the present invention;

FIG. 57 is an enlarged sectional view of a portion of the embodiment of FIG. 56;

FIG. 58 is a photographic representation of typical Prior Art AO-style bone screw;

FIG. 59 is a photographic representation of a bone screw of the present invention;

FIG. 60 is a diagram showing the experimental setup of a comparison between the two screws shown of FIG. 58 and FIG. 59;

FIG. 61 is a photographic representation showing the effect is the displacement experiment described in FIG. 60; and

FIG. 62 is a graph of the force versus displacement result of the displacement experiment described in FIG. 60.

### DETAILED DESCRIPTION OF THE DRAWINGS

The present inventors have identified shortcomings in bone implant devices of the prior art, and upon identification of the problems with the prior art, have provided a bone implant device which overcomes the problems of the prior art.

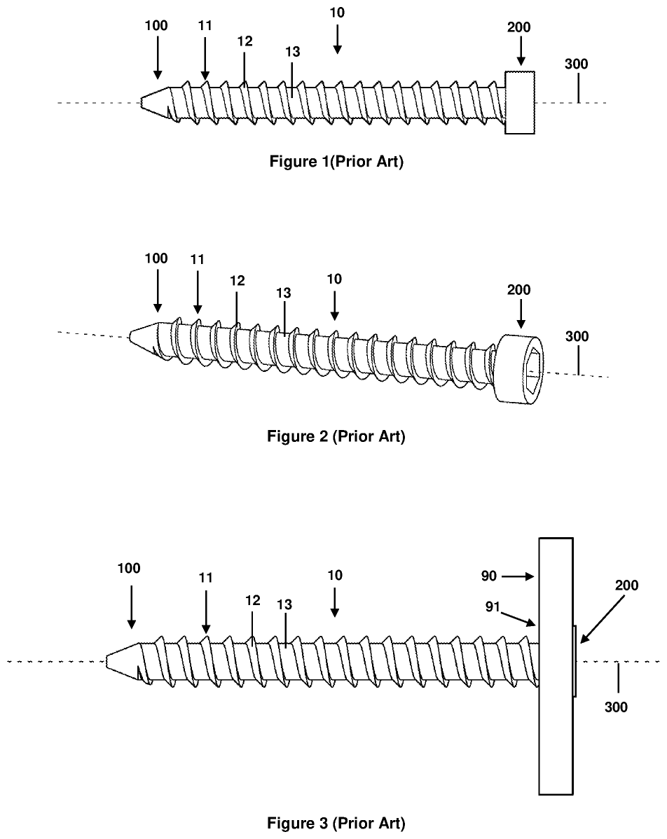

For comparative purposes, a typical bone implant device, in this case a bone screw embodying features of the prior art is first evaluated as described with reference to FIGS. 1 to 12, followed by which analysis and evaluation of a bone implant of the same type and of the same overall geometry and boundary conditions and embodying features of the present is conducted, in order to demonstrate the advantages and benefits as provided by the present invention.

Referring to FIGS. 1 to 3, 5, and 7 to 12 there is illustrated an orthopaedic implant device 10 which is a bone screw of the Prior Art used for fixing fractured or fragmented bone so that fragmented or fractured bone may be reduced to their correct anatomical positions while osteosynthesis, or bone healing, takes place.

The implant device 10 includes a distal end 100 for insertion into bone tissue, and a proximal end 200 that is operated or manipulated by a surgeon, and a central longitudinal axis 300 that extends from proximal to distal direction. The implant device 10 further includes a thread portion 12 comprised of a helical thread 11 having a buttress profile that follows a helical path around central shaft 13 of the implant device 10.

The implant device 10 may be formed from a biocompatible and corrosion-resistant metal alloy, preferably stainless steel, titanium or cobalt-chromium alloy. The implant device 10 may alternatively be formed from a biocompatible rigid or semi-rigid polymeric material suitable for orthopaedic implants and applications, such as polyether ether ketone (PEEK)

Further, the implant device 10 may also be formed from a biocompatible rigid or semi-rigid ceramic material suitable for orthopaedic implants, such as silica or hydroxyapatite-based ceramic materials.

Referring to FIG. 3, there is shown a sectional view of a portion of the implant device 10. The thread portion 12 includes a proximal facet 16, a crest 15, and a distal facet 14.

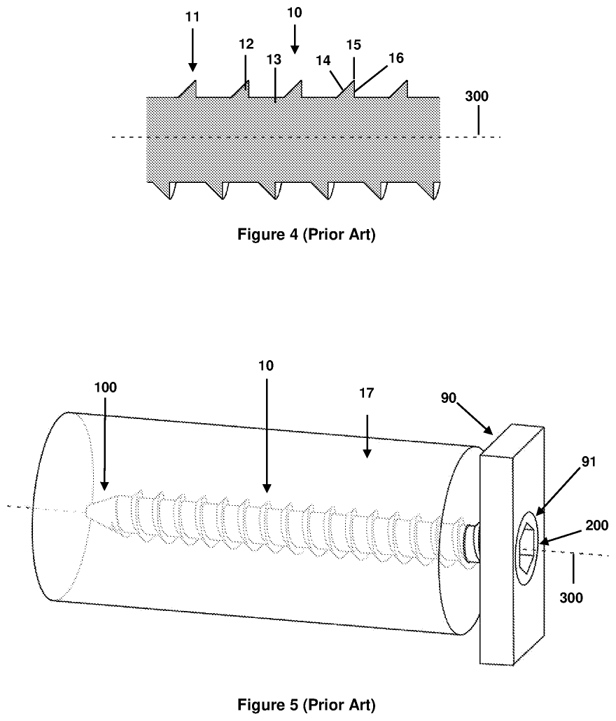

As shown in FIGS. 4, 5 and 6, the proximal end 200 of implant device 10 may be permanently or removably attached to a further device 90 such as bone plate, intramedullary nail, or other member, which may possess one or more holes 91 extending therethrough.

The implant device 10 may be attached to the fixation device 90 by first passing the distal end 100 of the implant device 10 through one such hole 91 and advancing the implant device 10 into bone tissue 17 until the proximal end 200 engages with the further device 90, such as through threads or sloped surfaces on 200 that mate with matching threads or sloped surfaces on hole 91.

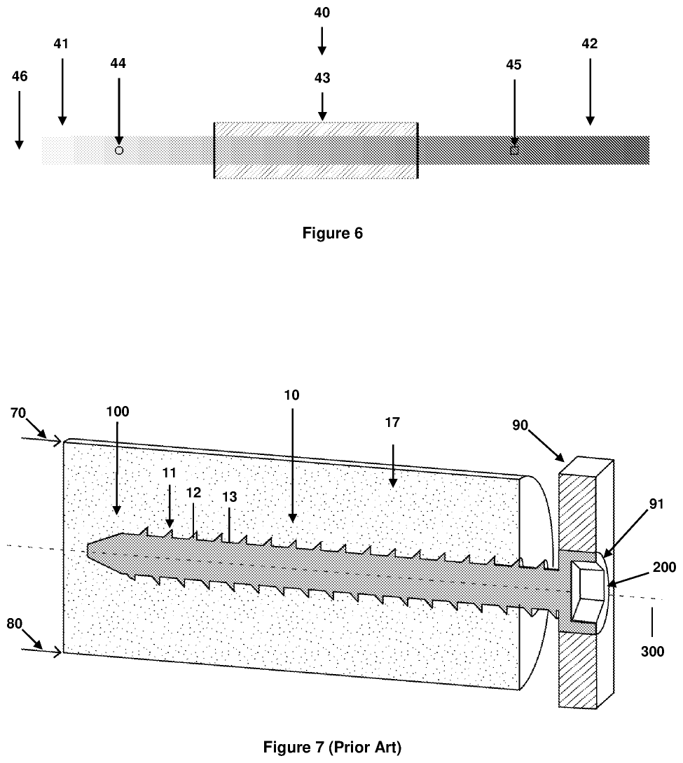

FIG. 6 illustrates a generic scalar number line measuring stress 40 applied to a small portion of bone tissue 17, showing the range 43 of stress that is applied to this portion of bone tissue 17 under physiological conditions.

Bone tissue stresses 44 with magnitudes in the range 41 from zero 46 to below the minimum extent of the physiological range 43, are insufficient to stimulate healthy biological activity in the bone tissue through the mechanobiological transduction process known as Wolff's Law. This can lead to bone resorption and/or aseptic loosening of implants in cases of chronic underexposure to stress, such as stress shielding of bone in the proximity of implants, and has been widely reported in then scientific literation as contributing to aseptic loosening, and bone and implant failure.

Bone tissue stresses 45 with magnitudes in the range 42 exceeding the physiological range 43, are known to cause mechanical damage the bone tissue, such as by compaction or tearing This reduces the structural integrity of the bone tissue and/or disrupts its normal biological activity and function, likewise leading to undesirable events such as implant loosening, migration, and/or cut-out, and implant system or implant failure.

The problems of the prior art as identified by the present inventors are demonstrated in FIGS. 7 and 9, and FIGS. 8 and 10.

As shown in FIGS. 7 and 9, there is shown a perspective longitudinal sectional and a longitudinal sectional view respectively of the implant device of FIGS. 1 to 6 engaged with bone tissue 17 and a further device 90. the section

The bone tissue 17/implant device 10/further device 90 system as depicted in FIGS. 7 and 9 is shown in a non-loaded state, with no physiological or external loading applied thereto.

For discussing and illustrating the position of the bone tissue 17 relative to the implant device 10, there may be considered to be datum lines 70 and 80 that are parallel to implant device's longitudinal central axis 300, and positioned to correlate with the top and bottom extents, respectively, of the bone tissue 17 in its initial position following insertion of implant 10.

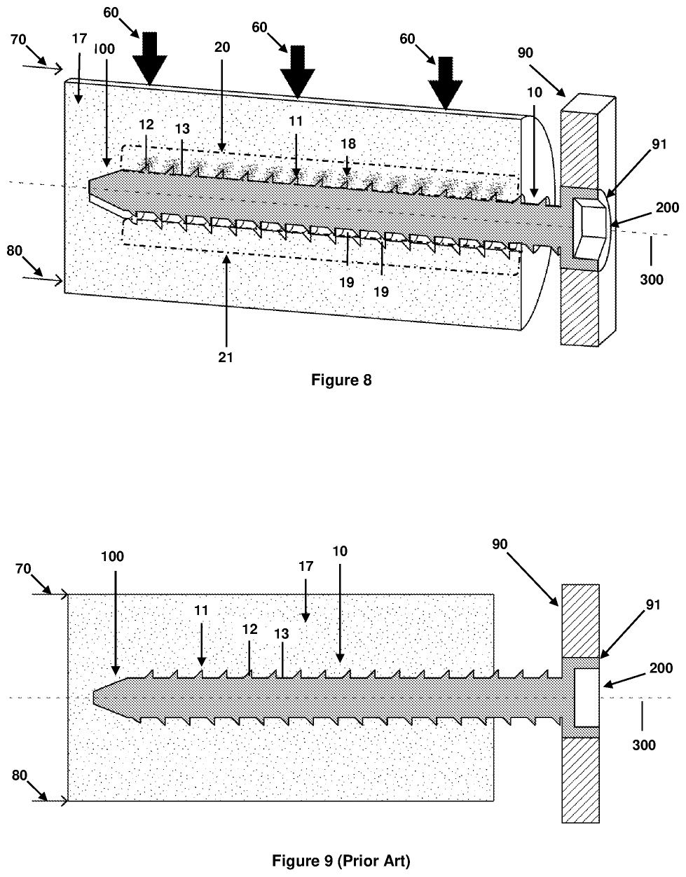

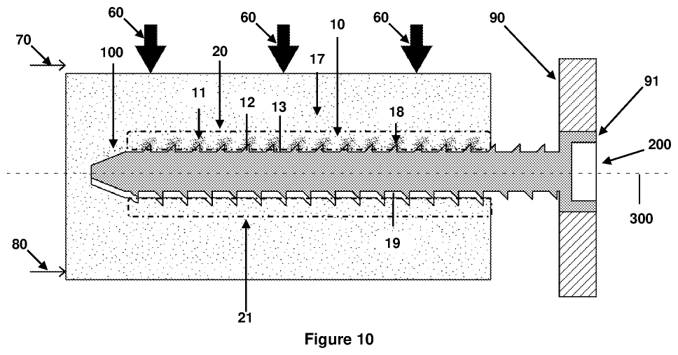

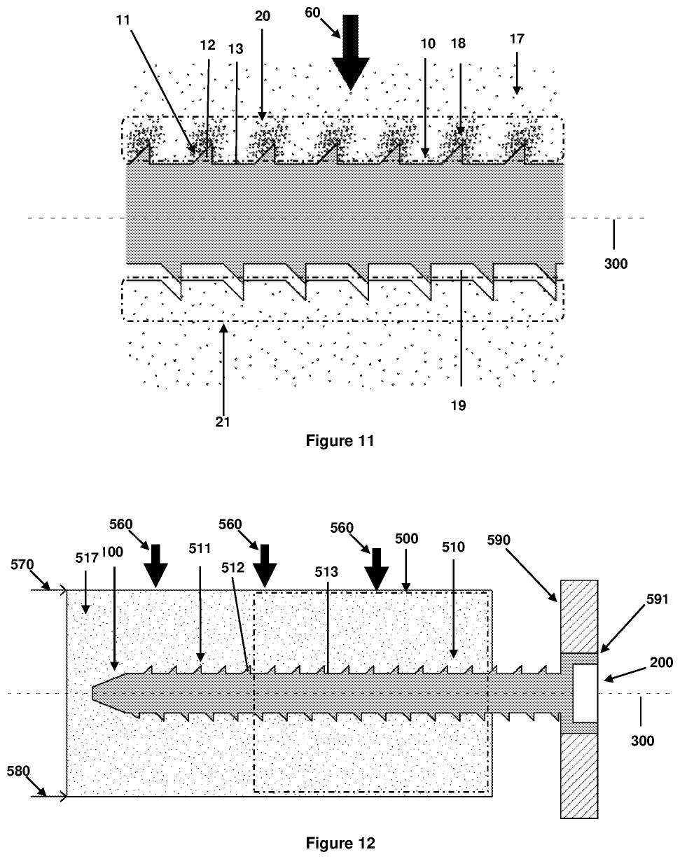

As shown in FIGS. 8 and 10, which correspond to FIGS. 7 and 9 referred to above, depict the bone tissue 17/implant device 10/further device 90 system following physiological loading applied thereto.

Following insertion of the implant device 10 into bone tissue 17, there may occur physiological or traumatic loading of the bone tissue 17 which urges the bone tissue 17 along a vector with a directional component at least partly perpendicular to the central longitudinal axis 300 of the implant device, depicted in FIGS. 8 and 10 as those force components of load 60 with a direction from datum line 70 to datum line 80.

The implant 10 and further device 90, which may be for example a bone plate, intramedullary nail, or other member 90 may be considered to be fixed in position in the reference frame of the present diagram, such that load 60 is the difference in loading forces applied to the bone tissue 17 and the implant device 10.

By the system being so urged by force components 60, a region 20 of the bone tissue 17 adjacent to the side of 10 that is predominantly facing the direction from which the load 60 originates, is compressed against an adjacent portion of the helical thread 11 and central shaft 13 of the implant device 10.

Being so compressed, stress concentrations 18 with magnitudes 42 exceeding the physiological range 43 as referred to in FIG. 6 above may form in those bone tissue portions 20 adjacent the implant such that they undergo damage in the form of undesirable compression, cracking, and/or compaction. Being so damaged, these bone tissue portions 20 may be of insufficient structural integrity to support further or such loading, leading to collapse of the bone tissue portions 20 and displacement of the bone tissue 17 relative to the implant device 10 as shown by the displacement of the top and bottom extents of 17 below their original datum lines of 70 and 80, respectively, whereby the implant device 10 and bone tissue 17 are displaced relative to each other.

Furthermore, exposure of bone portions 20 to excessive stress concentrations 18 may also lead to undesirable mechanobiological effects such as the disruption to bone remodeling activity, necrosis, and bone resorption, and the associated effects thereof as discussed above.

Concurrent with the compression of bone tissue region 20 due to the urging of bone tissue 17 by load 60, bone tissue in the region 21 of bone tissue 17 that is positioned roughly mirrored to 20 across the implant central longitudinal axis 300 may likewise be urged in the direction of 60 such that bone tissue in region 21 is relieved of existing compressive stresses, such as from elastic energy stored in the bone tissue during insertion of implant device 10 into bone tissue 17, and/or bone tissue in region 21 is displaced sufficiently such that the portions of bone in bone tissue region 21 that were previously in direct contact and engagement with the implant 10 are separated from the implant device 10, thus creating void spaces 19 between bone and the implant device 10.

Over time and progressively, the application of stress 44 of insufficient magnitude 41 to 21 in reference to FIG. 6 above may lead to undesirable bone loss in bone tissue region 21, ultimately resulting in aseptic loosening of implant device 10, including through the resorption of adjacent bone material by a mechanobiological effect known as stress shielding.

Conversely, as will be understood by those skilled in the art, physiological loading 60 may be applied to the implant 10 and/or further member 90 such as a bone plate, intramedullary nail, or other member, while considering the bone tissue 17 to be held in a fixed position relative to the reference frame of the present diagram. In such a case, the relative positions of 18, 19, 20, and 21 would be mirrored across the central longitudinal axis 300 of the implant device 60.

Referring to FIG. 11, there is shown an enlarged sectional view depicting a section of a portion of the implant device 10 when used for fixing fractured or fragmented bone so that fragmented or fractured bone may be reduced in their correct anatomical positions while osteosynthesis, or healing, takes place, such as is depicted and described with reference to FIGS. 8 and 10.

The displacement of the implant device 10 relative to the bone tissue 17, and crushing of bone tissue portions 20 and creating void spaces 19 can be clearly seen.

Referring to FIG. 12 there is shown a graphical representation of an implant device 510, of the same characteristics as shown and described with reference to FIGS. 1 to 11 above, for evaluation within a three-dimensional finite element analysis (FEA) model for assessment of load transfer characteristics to adjacent bone material.

FIG. 12 illustrates the initial conditions, prior to loading, of the three-dimensional finite element analysis (FEA) model constructed utilising mechanical simulation software, used to simulate the stress applied to bone tissue adjacent to an orthopaedic implant such as the implant device 510.

The FEA simulation includes the model implant device 510 of the type used for fixing fractured or fragmented bone so that fragmented or fractured bone may be reduced in their correct anatomical positions while osteosynthesis, or healing, takes place.

The FEA simulation was conducted use the software ABAQUS(6.13/CAE, Simulia, Providence, USA). The simulated implant material utilised was stainless steel with a Young's Modulus of 200 GPa and a Poisson's Ratio of 0.3 applied.

The simulated bone tissue was that representative of healthy human trabecular bone with a Young's Modulus of 260 MPa and a Poisson's Ratio of 0.29 applied.

The model implant device 510 has a clinically-relevant approximate length of 40 mm and diameter of 4.5 mm.

The implant device 510 model includes a distal end 100 that is placed in simulated bone tissue material 17 that possesses mechanical characteristics similar to human bone tissue.

The model implant device 510 includes a proximal end 200 similar to that which is operated by a surgeon in the case of a physical implant, and a longitudinal central axis 300 that follows a proximal to distal direction. The plane of the section of the model was cut having a normal vector that is also normal to 300 and as such, may be considered a longitudinal section.

The model of implant 510 also has a thread portion 511 with a buttress profile 512 that follows a helical path around a central shaft 513 of the implant device 510. The proximal end 200 of the model of the implant device 510 is attached to bone plate 590, by way of a hole 591.

The implant device 510 and bone plate 590 are fixed in position relative to each other in the FEA simulation. The simulation also includes a simulated physiological load 560 of 250N applied to the bone tissue 517 which is designed to urge the simulated bone tissue 517 along a vector with a directional component at perpendicular to the longitudinal central axis 300 of the implant device 510, depicted here as following a direction from datum line 570 to datum line 580.

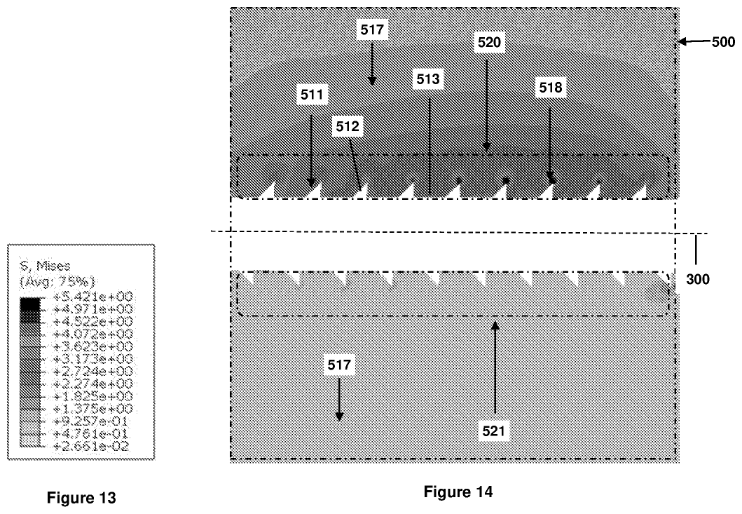

A window 500 is selected for depicting the stress field produced in the simulated bone tissue 517 during the FEA simulation. FIG. 13 illustrates the range of Von Mises stress depicted in FIG. 14 induced in the bone in the FEA simulation in MPa.

Referring to FIG. 14, there is shown the conditions following loading of the FEA model shown and described in reference to FIG. 12. Being so urged by load 560, a region 520 of the simulated bone tissue 517, adjacent to the side of the implant 510 that is predominantly facing the direction from which the simulated load 560 originates, is compressed against an adjacent portion of the modeled thread portion 511 and central shaft 513.

Being so compressed, stress concentrations 518 are shown with magnitudes in the simulated bone tissue portions 520, of a maximum magnitude of 5.4 MPa.

In a clinical application, exposure of the real equivalents of these bone portions 520 to high stress concentrations 518 can lead to damage of the bone tissue 517 in the form of undesirable compression, cracking, and/or compaction, ultimately contributing to implant migration within the real bone tissue, as well as undesirable mechanobiological effects such as the disruption of bone remodeling activity, necrosis, and bone resorption.

Concurrent with the compression of the simulated bone tissue region 520 due to the urging of bone tissue 517 by load 560, simulated bone tissue in the region 521 of 517 that is positioned roughly mirrored to 520 across the implant longitudinal central axis 300 is shown to be exposed to minimal stress as is shown from FIG. 14.

In a clinical application, chronic application of low levels of real stress 44 of insufficient magnitude 41 referring to FIG. 6, can lead to undesirable bone loss in bone tissue, causing aseptic implant loosening through the resorption of bone material by a mechanobiological effect of stress shielding and associated complaints as discussed above.

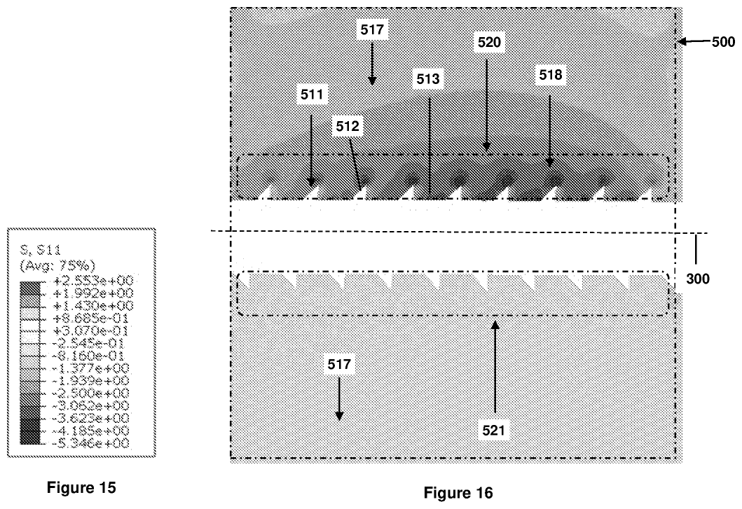

Referring to FIG. 15, there is shown the range of vertical principal stress depicted in FIG. 1 16 from FEA analysis of the model of FIG. 12, with a positive stress being equivalent to the upward direction and a negative stress being equivalent to the downward direction.

FIG. 16 illustrates the conditions following loading of the model of FIG. 12. Being so urged by load 560, region 520 of the simulated bone tissue 517, adjacent to the side of implant device 510 that is predominantly facing the direction from which the simulated load 560 originates, is compressed against an adjacent portion of the modeled thread portion 511 and central shaft 513.

Being so compressed, stress concentrations 518 of stress are shown with magnitudes in the simulated bone tissue portions 520, of a maximum magnitude of 2.55 MPa. Again, in a clinical application, exposure of the real equivalents of these bone portions 520 to high stress concentrations 518 again may lead to damage in the form of undesirable compression, cracking, and/or compaction, ultimately contributing to implant migration within the real bone tissue, as well as undesirable mechanobiological effects such as the disruption of bone remodeling activity, necrosis, and bone resorption.

Concurrent with the compression of the simulated bone tissue region 520 due to the urging of implant device 517 by load 560, simulated bone tissue in the region 521 of bone tissue 517 that is positioned roughly mirrored to 520 across the implant longitudinal central axis 300 is shown to be exposed to minimal stress.

Again, in a clinical application, chronic application of low levels of real stress 44 of insufficient magnitude 41 with reference to FIG. 6, may again lead to undesirable bone loss in bone tissue, causing aseptic implant loosening through the resorption of bone material by a mechanobiological effect known as stress shielding.

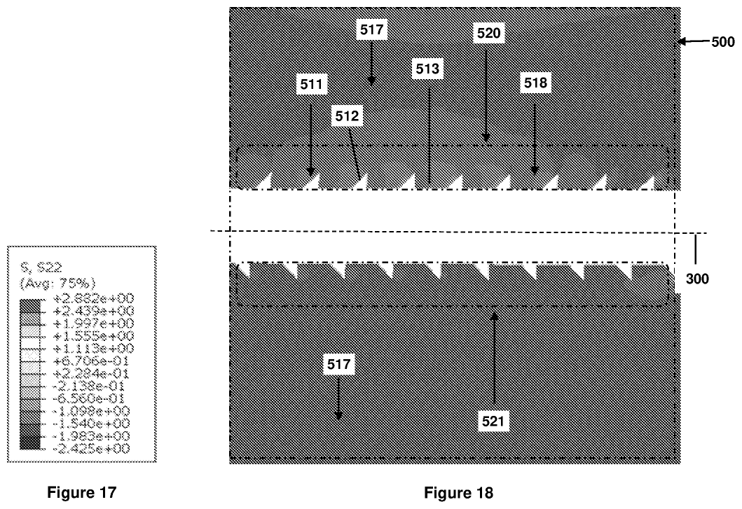

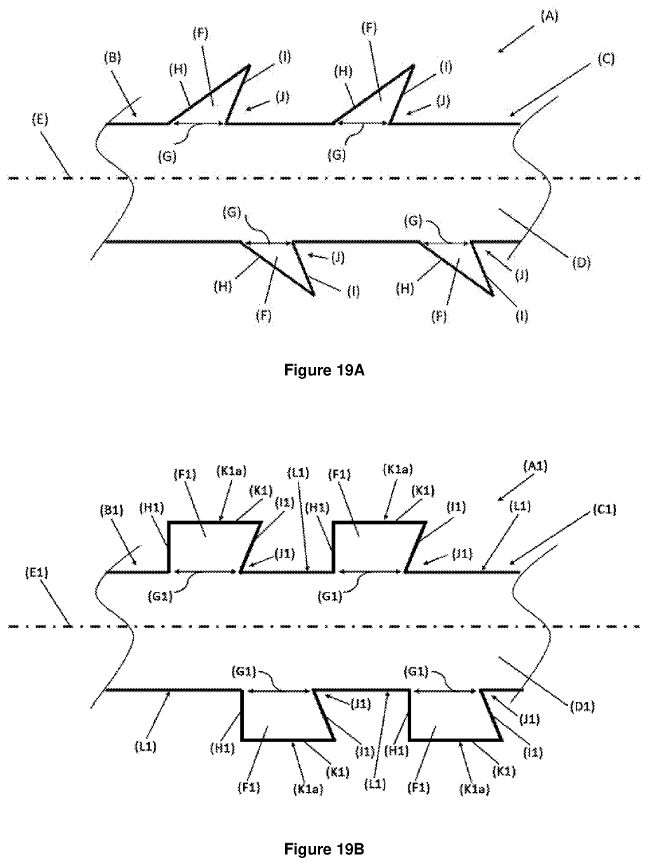

FIG. 17 illustrates the range of horizontal principal stress from the FEA model output of FIG. 18, where positive stress being equivalent to the rightward direction and negative stress equivalent to the leftward direction.

FIG. 18 illustrates the conditions following loading of the model shown in FIG. 12. Again being so urged by load 560, a region 520 of the simulated bone tissue 517, adjacent to the side of implant device 510 that is predominantly facing the direction from which the simulated load 560 originates, is compressed against an adjacent portion of the modeled 511 and central shaft 513.

Again being so compressed, small stress concentrations 518 are shown with magnitudes in the simulated bone tissue portions 520, of a maximum magnitude of 2.8 MPa. Concurrent with the compression of the simulated bone tissue region 520 due to the urging of bone tissue 517 by load 560, simulated bone tissue in the region 521 of bone tissue 517 that is positioned roughly mirrored to 520 across the implant device longitudinal central axis 300 is shown to be exposed to minimal stress.

As has been identified by the present inventors, an implant device of the bone screw type having a buttress thread, provides several biomechanical disadvantages:
(i) Excessive bone loading at portions of bone adjacent thread portions on a first side of the implant device,
(ii) Insufficient loading of bone to the second side of the implant device, and
(iii) Separation at the bone—implant interface of the second side of the implant device.

Excessive localised bone loading can cause localised bone damage from crushing of bone material.

Stress shielding due to insufficient bone loading results in bone resorption due to a mechanobiological effect on bone.

Collectively and individually, both excessive and insufficient loading to bone adjacent can exacerbate detrimental effects on surrounding bone tissue, resulting in;
Aseptic loosening,
Implant migration through bone,
Failure of an implant/bone fixation or maintenance system.
Catastrophic failure of bone material and implant devices.

This can lead to undesirable bone loss in bone tissue, causing aseptic implant loosening through the resorption of bone material by a mechanobiological effect of stress shielding and associated complaints as discussed above.

Whilst the FEA model utilised to provide the above observed phenomena is directed to a single static loading, as is known by those skilled in the art, FEA modelling is a useful analytical tool for biomechanical systems, implant and bone.

The observed deficiencies of such a fixation device having a buttress thread which is commonly used within the field of orthopaedics as identified by the present inventors is considered demonstrative of the clinical bone/implant environment.

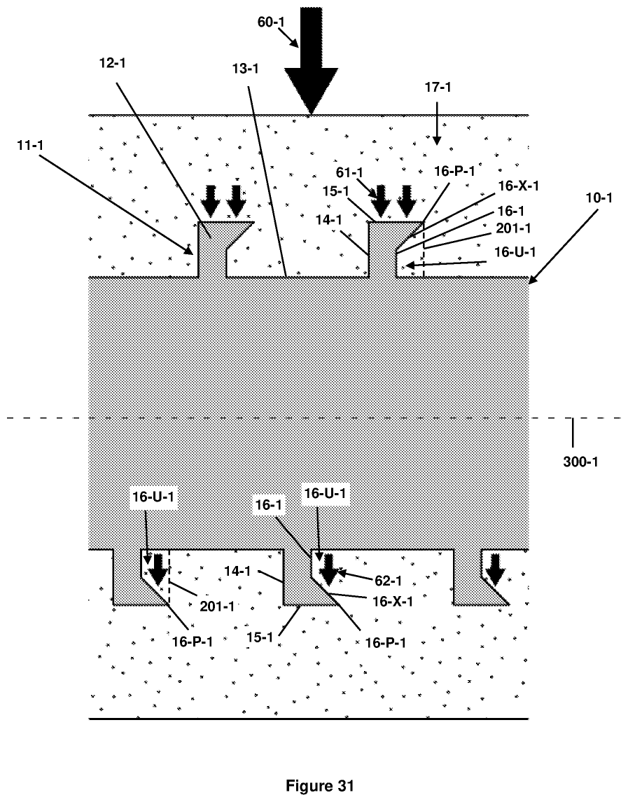

The present invention is now described with reference to FIGS. 19 to 62, whereby an embodiment of the present invention is provided with the same general geometric, size and mechanical properties to that of the Prior Art bone screw of FIGS. 1 to 18, and analysis conducted and performed using the same FEA model and characteristics for comparative purposes and consistency of analysis.

Referring to FIG. 19A, there is shown a longitudinal sectional schematic view of a portion of an implant device (A) according to the present invention. The present invention provides an implant device (A) for engagement with a bone of a patient. For example, the implant device (A) could be a bone screw, implant, suture anchor or the like.

The implant device (A) has a distal end (B), a proximal end (C), a central shaft (D) extending therebetween (B) and a longitudinal central axis (E).

The implant device (A) further includes a helical thread portion (F) which extends circumferentially about the central shaft (D) and extending from the distal end (B) towards the proximal end (C) thereof, and has a root (G) at the base of the helical thread portion (F) adjacent the central shaft (D).

The helical thread portion (F) includes a leading edge (H) and a trailing edge (I) both extending at least radially outwardly from the central shaft (D) and defining the thread portion (F) therebetween, with the root (G) of the helical thread portion (F) defined therebetween in a direction of the longitudinal central axis (E) of the implant device (A)

The leading edge (H) faces in a direction of at least towards the distal end (B) of the implant device (A), the said trailing edge (I) faces at least in a direction of towards the proximal end (C) of the implant device (A).

A portion of the trailing edge (H) extends in a direction towards the proximal end (C) of the device (A) further than the root (G) of the thread portion (F) such that said portion of the trailing edge (I) forms a recess (J) or “undercut” between the central shaft (D) and the trailing edge (I).

As is shown, there is an over-hang of the thread in the proximal aspect, which causes a recess under the thread which accommodates bone tissue therein when the implant device (A) is engaged with bone tissue of a subject.

Although the leading edge and the trailing edge are depicted as being linear, in other and alternate embodiments these may have varying surface geometries and shapes, and need not necessarily be linear.

FIG. 19B shows a further sectional schematic side view of a portion of an implant device (A1) according to the present invention, further illustrating the principles and further features of the present invention.

The features (A1) to (H1) of the present FIG. 19B correspond to features (A) to (H) of FIG. 19A and as such, the full description of all feature (A1) to (H1) are not repeated in reference to FIG. 19A.

The implant device (A1) again is for engagement with a bone of a subject, said implant device comprising a distal end (B1), a proximal end (C1), and a central shaft (D1) extending therebetween and a longitudinal central axis;

The implant device (A1) includes a helical thread portion (F1) which extends circumferentially about the central shaft (D1) and extends from the distal end (B1) towards the proximal end (C1), and has a and a root (G1) at the base of the helical thread portion (F1) adjacent the central shaft (D1).

The helical thread portion including a leading edge (H1) and a trailing edge (11) both extending at least radially outwardly from the central shaft (D1) and defining the thread portion (F1) therebetween.

The root (G1) of the thread portion is defined therebetween leading edge (H1) and a trailing edge (11) in a direction of the longitudinal central axis of the implant device (A1).

The leading edge faces (H1) in a direction of at least towards the distal end (B1) of the implant device Al), and said trailing edge (11) faces at least in a direction of towards the proximal end (C1) of the implant device (A1).

The implant device further includes a crest portion (K1) at the crest of the thread portion (F1).

The thread portion (F1) extends in at least a direction of from the distal end (B1) towards the proximal end (C1) and provides a recess (J1) between the central shaft (D1) and the thread portion (F1) for abutment and engagement with bone adjacent the thread portion.

The crest portion (K1) forms a radially outward portion of the thread portion (F1) and includes an engagement surface (K1a) for abutment and engagement with bone of a subject radially disposed from the thread portion (F1).

Upon engagement with radially disposed bone adjacent the thread portion (F1),the crest portion (K1) provides for distribution of stress induced in the bone adjacent the crest portion (K1) along said engagement surface (K1a), and the engagement surface (K1a) provides for reducing stress concentration in bone adjacent the crest portion (K1).

The recess (J1) is sized and shaped such that upon the implant device (A1) and adjacent bone in which the implant device (A1) is embedded being urged towards each other on a first side of the implant, which may be considered the upper side of the implant device as shown in FIG. 19, at least a portion of recess (F1) is urged against bone disposed within the recesses (F1) on the opposed side of the implant device which can be considered to be the lower side of the implant device (A1).

As is shown, the crest portion (F1) has a greater longitudinal length than that of the root portion (G1) in the direction of the longitudinal central axis (E1) of the implant device (A1).

In the present example, the leading edge (H1) is vertical, however in other embodiments it need not necessarily be so, and could be sloping or inclined, or of a curved shape. Alternatively, the leading edge (H1) could be provided for by a plurality of facets.

Similarly, the trailing edge (11) could be provided by a plurality of facets, or be curved, or varying geometry.

In the present embodiment, the leading edge (H1), the trailing edge (11) and the engagement surface (K1a) are all linear and clearly separate features.

However, in other or alternate embodiments, they could be curved, straight or combinations thereof.

The engagement portion (K1a) of the crest portion (IK1) could be least partially provided by the leading edge, or least partially provided by the trailing edge (i1), or a combination of both.

The crest portion (K1) itself may be comprised of one or more facets, again which may have flat or curved surfaces or combinations thereof.

Transition between the crest portion (K1) and the leading edge (H1) and/or the trailing edge (I1) may at a point or a crease, an edge, a facet, chamfer or other structural or geometric feature.

As will be noted, the crest portion (K1) is, at least in part, extends over the recess (J1) which is formed between the central shaft (D1) and the trailing edge (I1).

At least a portion of the crest portion (K1) provides for abutment with adjacent bone material and provides a surface for bearing against such bone for bearing physiological loads.

The crest portion (K1) can provide an increased longitudinal surface area, with respect to the longitudinal axis (E1) of the implant device (A1).

As can be seen, the recess (J1) in combination with the crest portion (K1), the longitudinal contact area of the implant device (A1) may be considered to be cumulative areas of the surfaces (L1) of the shaft (B1) between the roots (G1) of adjacent thread portions (F1), the surface of the crest portion (K1) as well as a horizontal portion or component of the leading edge (H1).

Additionally, the surface area of the trailing edge (I1), in some embodiments, may also contribute to providing addition surface area contact with adjacent.

As can be understood, by virtue of the engagement surface (K1a) being disposed radially outwardly from the longitudinal axis (E1) of the implant device (A1), this advantageously provides even further longitudinal contact area, due to the contact area being essentially circumferential, and being disposed at an outward radius, as should now be understood

Such an increased surface area as provided by the present invention, allows for stress fields in bone adjacent to the implant device (A1) which provides improved load transfer, in particular lateral load transfer between the implant device and adjacent bone, by way of the novel thread portion of the implant device.

Accordingly, the engagement surface (K1a) provides for stress concentration reduction adjacent the crest portion (K1) and localised load spreading across the engagement surface (K1a).

Reduction in localised excessive stress concentrations, for reasons including those as discussed further below, is advantageous for initial fixation, preventing or reducing bone loss and bone resorption, for accommodating subsequent physiological loading and reducing the incidence of migration of the implant relative to bone tissue, preventing subsequent aseptic loosening and implant catastrophic failure due to lack of bone stock support.

The recess (J1), as discussed above, provides advantages of:
(i) Increased bone contact area adjacent the threat portion (F1)
(ii) In the case of loading including lateral loads relative between the implant device and adjacent bone, on the tensile side (i.e. the side whereby there is little or no compressive loading between the implant device and adjacent bone, bone within the recess (J1) as the effect of applying load to adjacent bone disposed within the recess (J1), and may also in some embodiments assist in reducing localised excessive stresses in bone tissue adjacent the recesses (J1) in the compressive side of the implant device, which:
   a. Assists in opposing migration of the implant relative to adjacent bone tissue, and
   b. Provides physiological advantages of providing loading to adjacent tissue, again for initial fixation, preventing or reducing bone loss and bone resorption, for accommodating subsequent physiological loading and reducing the incidence of migration of the implant relative to bone tissue, preventing subsequent aseptic loosening and implant catastrophic failure due to lack of bone stock support.
(iii) Furthermore and advantageously, the provision of the recess (J1) provides for:
   a. less removal of bone, which provides less trauma to the bone tissue and allows more to remain for bone growth and physiological restoration, and
   b. thus allows more bone tissue to remain in contact for greater initial fixation and stability as well as load and stress transfer between bone and the implant, and the physiological advantages associated therewith.

Together, recess (and (J1) and the crest (K1) of the present invention, provide for improved load transfer characteristics between the implant and adjacent bone, and the present invention provides at least the advantages of:
(1) reducing excessive localised bone-damaging compressive stresses induced in bone material adjacent the thread portion of the implant device whilst providing a more uniform load transfer profile,
(2) inducing localised stress in bone material adjacent the thread portion of the implant device in regions whereby negligible load is imparted to such adjacent bone, and
(3) assisting in opposing migration of the implant device relative to adjacent bone

As provided by the present invention, the advantages provided by the present invention include providing a preferred localised stress environment which prevents or reduces localised trauma to bone tissue, and induced localised stresses to as to prevent or reduce bone resorption due to stress shielding, which assists in:
maintaining integrity of the bone/implant interface and stability of the bone/implant system,
reducing migration of the implant device through bone tissue,
reducing movement of the implant relative to adjacent bone tissue,
reducing bone loss through stress shielding and crushing and damage to bone adjacent the implant device, and

preventing aseptic loosening, which may precipitate major implant/system failure, or bone or implant failure. As follows, the present invention and embodiments thereof are described and compared to those of the prior art as discussed above on reference to FIGS. 1 to 18 for comparative purposes for exemplifying the benefits and advantages of the present invention in comparison with those of devices of the prior art.

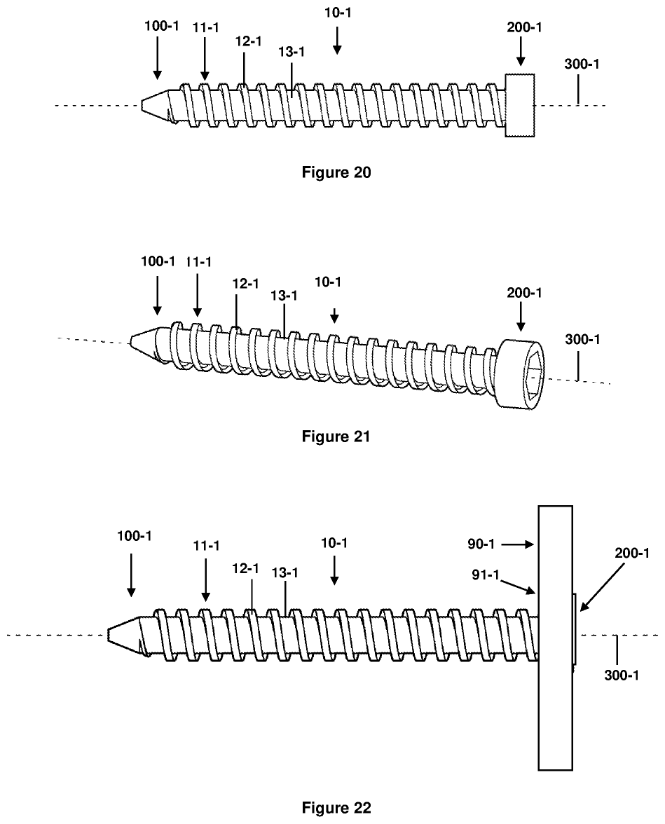

FIG. 20 and FIG. 21 show an embodiment of an implant device according to the present invention, with the implant device being an orthopaedic implant device 10-1 as used for fixing fractured or fragmented bone so that fragmented or fractured bone may be reduced in their correct anatomical positions while osteosynthesis, or healing, takes place.

Implant device 10-1 has a distal end 100-1 for inserting into bone tissue, and a proximal end 200-1 that is operated by a surgeon, and a central axis 300 that extends longitudinally in a proximal to distal direction.

Implant device 10-1 further includes thread portions 11-1 with a square angled-undercut profile 12-1 in this embodiment that follows a helical path around a central shaft 13-1. The implant device 10-1 may be formed from a biocompatible and corrosion-resistant metal alloy, preferably stainless steel, titanium or cobalt-chromium alloy.

Alternatively, the implant device 10-1 may also be formed from a biocompatible rigid or semi-rigid polymer suitable for orthopaedic implants, such as polyether ether ketone (PEEK).

The implant device 10-1 may also be formed from a biocompatible rigid or semi-rigid ceramic material suitable for orthopaedic implants, such as silica or hydroxyapatite-based ceramics.

FIG. 22 shows the embodiment of the present invention as in FIG. 20 and FIG. 21, wherein the proximal end 200-1 of the implant device 10-1 may be permanently or removably attached to another member 200-1 such as a bone plate, intramedullary nail, which may possess one or more holes 91-1.

The implant device 10-1 may be attached to the other member 90-1 by first passing the distal end 100-1 through one such hole 91-1 and advancing the implant device 10-1 in until the proximal end 200-1 engages with 90-1, such as through threads or sloped surfaces on 200-1 that mate with matching threads or sloped surfaces on 91-1.

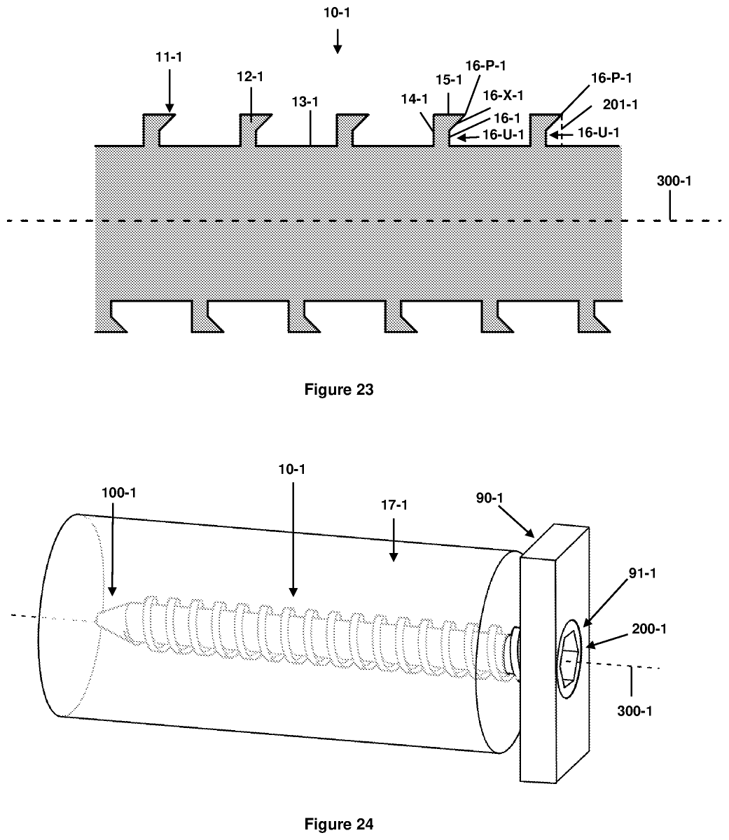

FIG. 23 illustrates an enlarged sectional view of the implant device of FIGS. 20 to 22 embodiment whereby the plane of the section cut has a normal vector that is also normal to 300-1, and the portion shown is roughly near the midpoint between 100-1 and 200-1.

The thread profile 12-1 of each thread portion 11-1 possesses a leading edge having at least a distal facet 14-1, a crest 15-1 that in the present embodiment, is generally flat or rounded and is also generally parallel to 300-1, an undercut 16-X-1 formed by the trailing edge that is a surface or curve that begins at the most proximal point of 15-1 and extends generally towards 300-1 or 100-1, and a proximal facet 16-1. The portions of crest 15-1 and undercut facet 16-X-1 that are nearest to the proximal end 200-1 of the implant 10-1 meet a connecting feature 16-P-1 which may be a point, edge, fillet, facet, chamfer or similar feature.

By projecting a datum line 201-1 from the most proximal portion of 16-P-1 towards 300-1 until reaching 13-1, an undercut void space 16-U-1 may be formed. In cases where 10-1 is inserted into bone tissue, this undercut void space 16-U-1 may be occupied by a portion of bone tissue.

Similarly as described with reference to FIG. 20, the trailing edge extends further from the root of the thread so as to form the undercut void space.

FIG. 24 illustrates the implant device embodiment of the present invention as in FIG. 22, which has been inserted into bone tissue 17-1. This bone tissue 17-1 may consist of a single bone, multiple nearby bones, collection of bone fragments, fractured bone, bony bodies, bone tissue, and/or fractured bones or bone tissue.

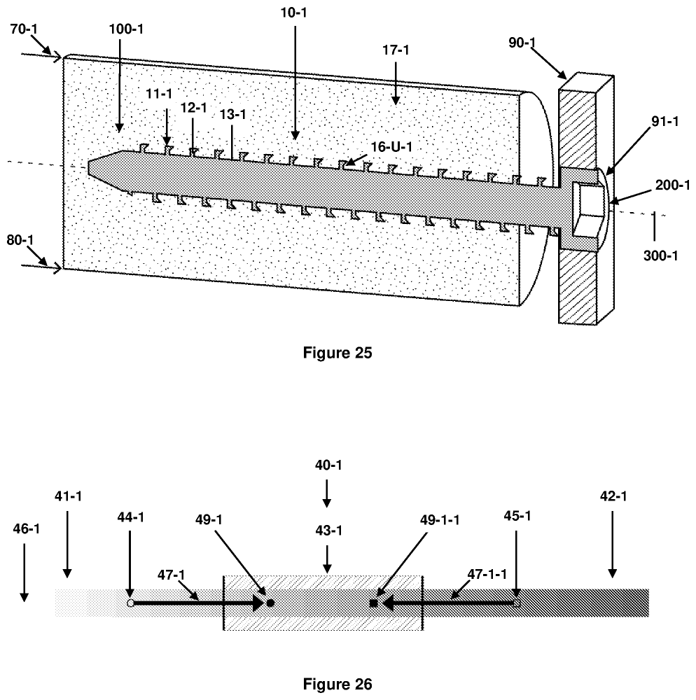

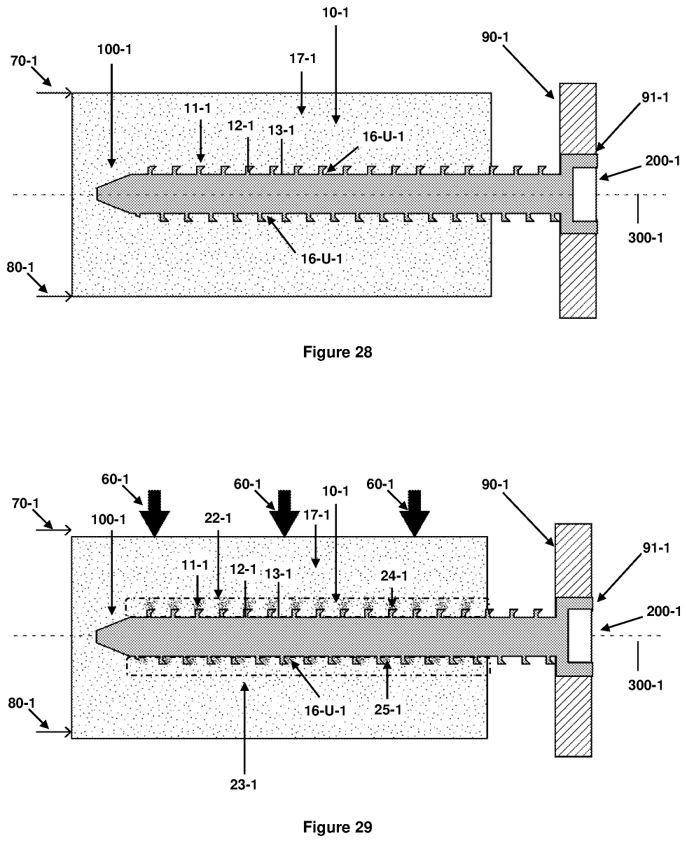

Referring to FIG. 25 and FIG. 28, there is shown a sectional view of implant device embodiment of the present invention as in FIG. 24. For discussing the position of the bone tissue 17-1 relative to the implant device 10-1, there may be considered to be datum lines 70-1 and 80-1 that are parallel to implant device's central axis 300-1, and positioned to match the top and bottom extents, respectively, of the bone tissue 17-1 in its initial position following insertion of implant 10-1.

As in FIG. 23, the thread profile 12-1 of each of one or more threads 11-1 forms an undercut void space 16-U-1. In FIG. 25, these undercut void spaces 16-U-1 are at least partly occupied by portions of the bone tissue 17-1.

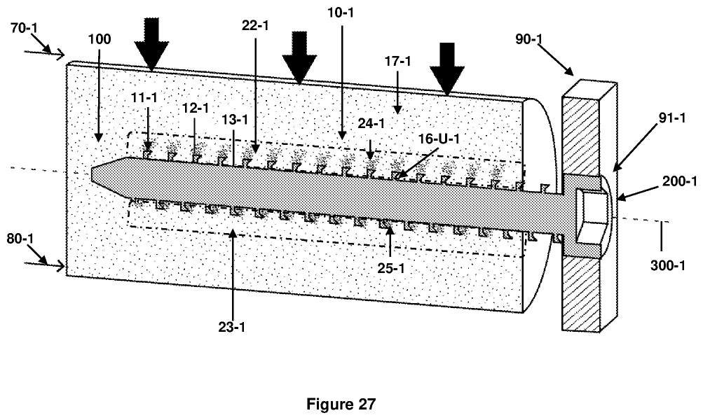

FIG. 26 illustrates a generic scalar number line measuring stress 40-1 applied to a small portion of bone tissue 17-1, showing the range 43-1 of stress that is applied to this portion of bone tissue 17-1 under physiological conditions.

Bone tissue stresses 44-1 with magnitudes in the range 41-1 from zero 46-1 to below the minimum extent of the physiological range 43-1, are insufficient to stimulate healthy biological activity in the bone tissue through the mechanobiological transduction process known as Wolff's Law, which can lead to bone resorption and/or aseptic loosening of implants in cases of chronic underexposure to stress, such as stress shielding nearby implants.

An orthopaedic implant may possess design features that are useful in reducing the rate of incidence of stress shielding in the bone tissue adjacent or nearby the implant. Such useful design features may provide a stress-increasing effect 47-1 on bone tissue having insufficient levels of stress 44-1, thereby increasing the total stress applied to said bone tissue at least partway to a magnitude 49-1 within the physiological range 43-1.

As in FIGS. 23, 24 and 25, at least one of the threads of any orthopaedic implant embodiments of the present invention, such as implant device 10-1, possesses an undercut facet 16-X-1, or a similar design feature, which provides a stress-increasing effect 47-1 as described in reference to FIG. 26 to bone tissue occupying the undercut void space 16-U-1 by compressing said bone tissue when the bone tissue is urged or displaced in a direction relative to 10-1 at least partially away from 300-1.

As shown FIG. 24 and FIG. 25, bone tissue 17-1 may be considered to consist of portions that are mechanically connected or at least in partial direct or indirect mechanical contact. Therefore, the application of force to any portion of 17-1 will directly or indirectly transmit some of that force to the remaining portions of 17-1. If, therefore, the stress-increasing effect 47-1 described above in reference to FIG. 26 is applied to a portion of bone tissue 17-1, we may assume that other portions of bone tissue 17-1 may be at least partly relieved of stress, contributing in part to a stress-relieving effect 47-1-1.

In particular, this will be the case when 47-1 is applied to bone tissue in the undercut void spaces 16-U-1 of bone tissue further than 300 from the origin of the force. In such a case, bone tissue on the side nearer than 300 to the origin of the force will experience at least a portion of the stress-relieving effect 47-1-1, particularly in the bone tissue adjacent to the thread crests 15-1.

Again referring to FIG. 26, bone tissue stresses 45-1 with magnitudes in the range 42-1 exceeding the physiological range 43-1, cause mechanical damage the tissue, such as by compaction or tearing, that reduces structural integrity of the bone tissue and/or disrupts its normal biological activity, likewise leading to undesirable events such as implant loosening, migration, and/or cut-out.

As shown FIG. 23 and FIG. 24 the orthopaedic implant 10-1, possesses a crest 15-1 that is generally flat or rounded and is also generally parallel to 300-1, or a similar design feature, which contributes at least in part to stress-decreasing effect 47-1-1 to bone tissue adjacent to the thread crests 15-1 when said bone tissue is urged or displaced in a direction relative to 10-1 at least partially towards 300-1.

The stress-relieving effect 47-1-1 may be sufficient to reduce the excessive stress 45-1 at least partway to a lower value 49-1-1 that is within the physiological range 43-1 again in reference to FIG. 26.

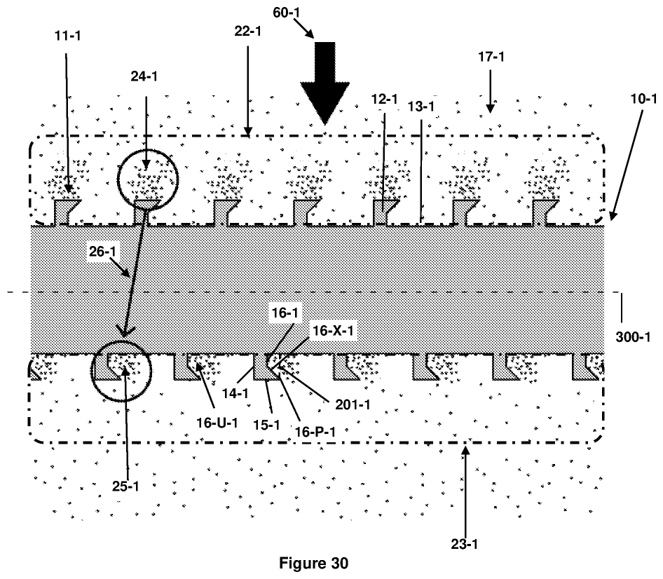

FIG. 27 and FIG. 29 shown sectional views of an orthopaedic implant embodiment of implant device 10-1 when inserted into and engaged with bone tissue 17-1, and whereby there may occur physiological or traumatic loading 60-1 of the bone tissue 17-1 which urges the bone tissue 17-1 along a vector with a directional component at least partly perpendicular to the central axis of the implant 300-1, depicted here as those force components with a direction from datum line 70-1 to datum line 80-1.

Similarly as in FIG. 25, the undercut void spaces 16-U-1 are of the threads 11-1 are at least partly occupied by portions of the bone tissue 17-1. The implant device 10-1 and further member 90-1 such as a bone plate, intramedullary nail, may be considered to be fixed in position in the reference frame of the present diagram, such that load 60-1 is the difference in loading forces applied to the bone tissue 17-1 and the implant 10-1.

Being so urged by load 60-1, a region 22-1 of the bone tissue 17-1, adjacent to the side of implant device 10-1 that is predominantly facing the direction from which the load 60-1 originates, is compressed against an adjacent portion of the threads 11-1 and shaft 13-1, forming concentrations of stress at least adjacent to the crests 15-1 within 22-1.

As in FIG. 26, the flat or rounded design of 15-1 may contribute at least in part with the relief of stress concentration 47-1-1 in bone tissue adjacent to 15-1 within 22-1.

Concurrent with the compression of bone tissue in the region of 22-1, bone tissue in the region 23-1 of 17-1 that is positioned roughly mirrored to 22-1 across the implant axis 300-1 may likewise be urged in the direction of load 60-1 due to the direct or indirect mechanical connection or contact between portions of 17-1 such that at least some of the portions of 23-1 within the undercut void spaces 16-U-1 are urged towards the thread undercut 16-X-1.

Being so urged, these portions of bone 23-1 may undergo a stress-increasing effect 47-1, as in FIG. 2 4, forming concentrations of stress 25-1 that are both beneficial to (1) reducing the incidence and severity of stress shielding, and also (2) contributory towards to the magnitude of the stress-relieving effect 47-1-1 within 22-1.

In the case of orthopaedic implant embodiments of the present invention, such as implant device 10-1, the complimentary beneficial effects 47-1 and 47-1-1 yielded by such thread design features as 15-1 and 16-X-1 may thereby reduce the magnitude of stress concentrations 24-1 in 22-1 from physiologically-excessive magnitudes 42-1 to the physiological range 43 with reference to FIG. 26, while simultaneously increasing the magnitudes of stress concentrations 25-1 in 23-1 from physiologically-insufficient magnitudes 41-1 to the physiological range 43.

This advantageous effect may thereby reduce undesirable effects such as damage to bone tissue, compression, cracking, compaction, structural weakening, aseptic loosening, implant migration, implant cutout, and similar deleterious phenomena related to excessive stress in 22-1, while reducing stress shielding, bone resorption, bone loss, aseptic loosening, and similar deleterious phenomena related to insufficient stress in 23-1, thereby contributing to a more firm fixation or anchorage of bone tissue by orthopaedic implant embodiments of the present invention and increased longevity of a bone/implant system, such as implant device 10-1.

The advantages and function, as described above in reference to the present embodiment and as described in reference to FIG. 19B whereby the advantages of a “crest portion” and a “recess” or undercut, as will be understood contribute to the advantages of the present embodiment as well as subsequent embodiments. Such features of a “crest portion” and a “recess” or undercut, further contribute to providing the overall mechanical and physiological advantages of an implant device as provided by the present invention and described above in reference to FIG. 19B, as well as described further below in respect of the present invention.

Conversely, as will be understood, physiological loading 60-1 may be applied to the implant 10-1 and/or a bone plate, intramedullary nail, or other member 90-1, while considering the bone tissue 17-1 to be held in a fixed position relative to the reference frame of the present diagram. In such a case, the relative positions of 18-1, 19-1, 22-1, 23-1 and their accompanying elements would be mirrored across the axis 300.

FIG. 30 shows a portion of an enlarged view of the orthopaedic implant device 10-1 embodiment of the present invention as in FIG. 27, shown in detail focused on a portion of the implant device 10-1 roughly at the midpoint between 100-1 and 200-1.

As in FIG. 27, the beneficial improvements to the thread design, including 15-1 and 16-X-1 contribute at least partly to an increase 47-1 of stress concentrations 25-1 in 23-1, and a decrease 47-1-1 of stress concentrations 24-1 in 22-1. The decrease 47-1-1 of stress in one portion of 17-1 occurs at least in part due to the increase of stress in other portions, as the portions of 17-1 may be considered at least partly mechanically connected or in partial mechanical contact.

Therefore, stress from the high stress concentration 24-1 regions of 22-1 may be considered to be transferred 26-1 to the lower-stress concentration 25-1 regions of 23-1.

FIG. 30 illustrates detail focused portion of an orthopaedic implant device 10-1 embodiment of the present invention as in FIG. 29. As the portions of bone 17-1 may be considered at least partly mechanically connected or in partial mechanical contact with one another, loading of load 60-1 applied to the bone tissue 17-1 is transferred concurrently to all threads 11-1 of 10-1 that are at least in at least partial contact with a portion of 17-1.

The load 60-1 is transferred at least in part to a set of partial forces that include but are not limited to the partial forces 61-1 applied by adjacent bone tissue to the crests 15-1 of those threads nearest to the origin of 60-1, and the partial forces 62-1 applied by adjacent bone tissue to the undercuts 16-X-1 of those threads furthest to the origin of 60-1.

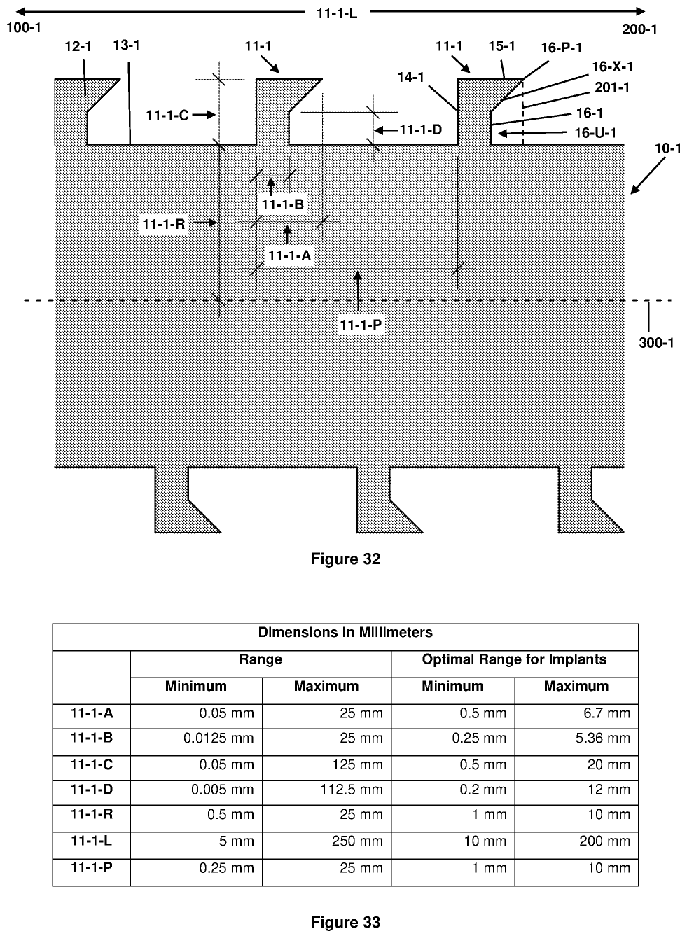

FIG. 32 shows an enlarged a sectional; view of the orthopaedic implant device 10-1 embodiment of the present invention as in FIG. 29.

The horizontal component of a line is, in FIG. 32, the component that is parallel to 300-1. The vertical component of a line is, in FIG. 32, the component that extends perpendicularly to 300-1.

All portions, lines, points, curves, edges, corners, fillets, chamfers, and other features that are used as references in the measurement of a dimension in FIG. 32 may be assumed to be on the same plane.

The dimension 11-1-A is the length of the horizontal component of a line extending from the most distal portion of a thread to its most proximal portion. The dimension 11-1-B is the length of the horizontal component of a line extending from the most distal portion of a thread to the most proximal portion of the portion of the thread adjacent to both 16-U-1 and 13-1.

The dimension 11-1-C is the length of the vertical component of a line extending from the portion of the thread that is most proximal to the portion of the thread that is furthest vertically from the shank 13-1.

The dimension 11-1-D is the length of the vertical component of a line extending from the shank 13-1 to the portion of 16-X-1 that is vertically closest to the shank 13-1. The dimension 11-1-R is the length of the vertical component of a line extending from 300-1 to the shank 13-1. The dimension 11-1-L is the length of the horizontal component of a line extending from the distal end 100-1 of the orthopaedic implant device 10-1 to its proximal end 200-1.

The dimension 11-1-P is the length of the horizontal component of a line extending from the most distal portion of a thread to the most distal portion of the next-most-proximal thread of 10-1. The dimensions shown in FIG. 32 may or may not be common for all threads in a single orthopaedic implant embodiment of the present invention.

Variable thread pitch and size, for instance, is a common feature of orthopaedic implants that will be familiar to those skilled in the art. These dimensions may be selectively tuned to appropriately address the requirements of a given anatomical position or application, such as decreasing 11-1-B and increasing 11-1-D so as to increase the size of 16-U-1 and thereby likewise increase the quantity of stress transferred 26-1, as in FIG. 30.

This is a useful feature of orthopaedic implants, as it permits designs to be produced that control the distribution of stress in adjacent bone tissue to prevent excessive or insufficient stress exposure.

FIG. 33 shows a range of possible values of the dimensions described in FIG. 32, as well as those values most likely to be optimal for orthopaedic implant applications.

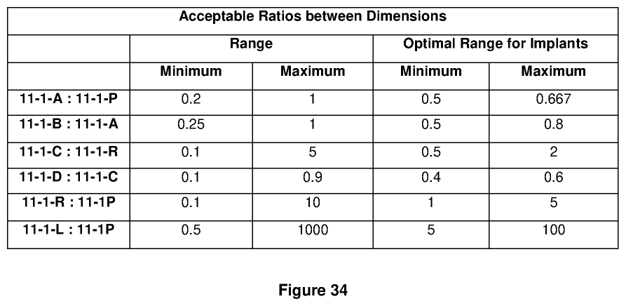

FIG. 34 shows a range of possible values of the ratios between the dimensions described in FIG. 23, as well as those values most likely to be optimal for orthopaedic implant applications.

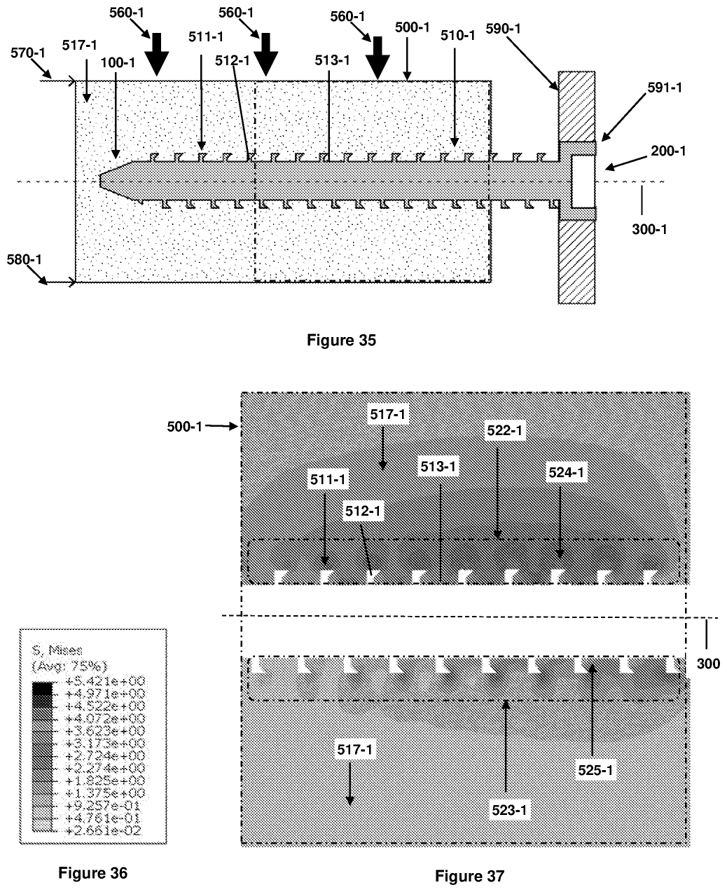

FIG. 35 illustrates the initial conditions, prior to loading, of a three-dimensional finite element analysis (FEA) model constructed in the mechanical simulation), used to simulate the stress applied to bone tissue adjacent to an orthopaedic implant device 500-1.

The FEA simulation was conducted use the software ABAQUS(6.13/CAE, Simulia, Providence, USA). The simulated implant material utilised was stainless steel with a Young's Modulus of 200 GPa and a Poisson's Ratio of 0.3 applied.

The simulated bone tissue was that representative of healthy human trabecular bone with a Young's Modulus of 260 MPa and a Poisson's Ratio of 0.29 applied.

The simulation includes a model orthopaedic implant device 510-1 used for fixing fractured or fragmented bone so that fragmented or fractured bone may be reduced in their correct anatomical positions while osteosynthesis, or healing, takes place.

This model implant 510-1 has a clinically-relevant approximate length of 40 mm and diameter of 4.4 mm, and possesses mechanical characteristics similar to stainless steel with a Young's Modulus of 200 GPa.

This implant device 510-1 model includes a distal end 100-1 that is placed in simulated bone tissue 17-1 that possesses mechanical characteristics similar to human bone tissue, with those bone tissue mechanical characteristics matching those of FIG. 10.

The model implant device 510-1 includes a proximal end 200-1 similar to that which is operated by a surgeon in the case of a physical implant, and a central axis 300-1 that follows a proximal to distal direction.

The plane of the section cut has a normal vector that is also normal to 300-1. The model of implant device 510-1 also has threads 511-1 with a buttress profile 512-1 that follows a helical path around central shaft 513.

The proximal end 200-1 of the model of implant 510-1 is attached to bone plate 590-1, by way of a hole 591-1. The implant 510-1 and bone plate 590-1 are fixed in position in the simulation. The simulation also includes a simulated physiological load of 250N applied to the bone 560 which is designed to urge the simulated bone tissue 517-1 along a vector with a directional component at perpendicular to the central axis of the implant 300-1, depicted here as following a direction from datum line 570-1 to datum line 580-1. A window 500-1 is selected for depicting the stress field produced in the simulated bone tissue 517-1 during the simulation.

FIG. 36 illustrates the range of Von Mises stress for the simulation described in FIG. 35.

FIG. 37 illustrates the conditions following loading of the model shown in FIG. 35, showing Von Mises stress using the scale in FIG. 36. Being so urged by 560-1, a region 522-1 of the simulated bone tissue 517-1, adjacent to the side of 510-1 that is predominantly facing the direction from which the simulated load 560-1 originates, is compressed against an adjacent portion of the modeled threads 511-1 and shaft 513-1.

Being so compressed, concentrations 518-1 of stress are shown with magnitudes in the simulated bone tissue portions 522-1, of a maximum magnitude of 3.17 MPa. In a clinical application, exposure of the real equivalents of these bone portions 522-1 to stress concentrations 524-1 of an acceptable physiological range would maintain bone health through mechanobiological stimulation as in Wolff's Law, while being less than the magnitude necessary to cause damage to bone tissue.

Concurrent with the compression of the simulated bone tissue region 522-1 due to the urging of 517-1 by 560-1, simulated bone tissue in the region 523-1 of 517-1 that is positioned roughly mirrored to 522-1 across the implant axis 300-1 is shown to be exposed to stress concentrations 525-1 of 2.27 MPa, resulting mainly from the entrapment of bone tissue within 16-U-1 by 16-X-1.

Exposure of bone tissue to such an acceptable physiological range would maintain bone health through mechanobiological stimulation as in Wolff's Law, while being less than the magnitude necessary to cause damage to bone tissue.

In a clinical application, the distribution of stress to across the bone tissue surrounding both the side facing a load and the side opposite may have utility in providing firm fixation of orthopaedic implants in bone while stimulating bone health and strength.

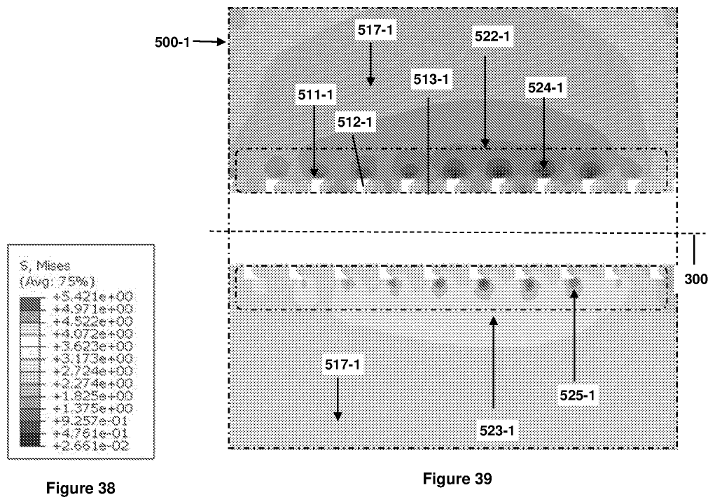

FIG. 38 illustrates the range of vertical principal stress depicted in FIG. 35, with positive being equivalent to the upward direction and negative being equivalent to the downward direction.

FIG. 39 illustrates the conditions following loading of the model shown in FIG. 35, showing vertical principal stress using the scale of FIG. 38. Being so urged by 560-1, a region 522-1 of the simulated bone tissue 517-1, adjacent to the side of 510-1 that is predominantly facing the direction from which the simulated load 560-1 originates, is compressed against an adjacent portion of the modeled threads 511-1 and shaft 513-1.

Being so compressed, concentrations 524-1 of vertical principal stress are shown with magnitudes in the simulated bone tissue portions 522-1 of a maximum magnitude of 4.18 MPa. In a clinical application, exposure of the real equivalents of these bone portions 522-1 to stress concentrations 524-1 of an acceptable physiological range would maintain bone health through mechanobiological stimulation as in Wolff's Law, while being less than the magnitude necessary to cause damage to bone tissue.

Concurrent with the compression of the simulated bone tissue region 522-1 due to the urging of 517-1 by 560-1, simulated bone tissue in the region 523-1 of 517-1 that is positioned roughly mirrored to 522-1 across the implant axis 300-1 is shown to be exposed to vertical stress concentrations 525-1 of 1.43 MPa, resulting mainly from the entrapment of bone tissue within 16-U-1 by 16-X-1. Bone tissue exposed to such an acceptable physiological range would maintain bone health through mechanobiological stimulation as in Wolff's Law, while being less than the magnitude necessary to cause damage to bone tissue.

In a clinical application, the distribution of stress to across the bone tissue surrounding both the side facing a load and the side opposite may have utility in providing firm fixation of orthopaedic implants in bone while stimulating bone health and strength.

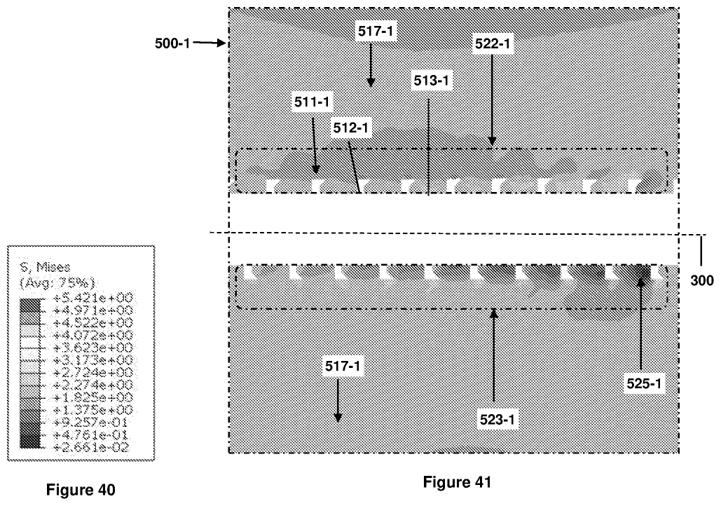

FIG. 40 illustrates the range of horizontal principal stress depicted in FIG. 35, where positive being equivalent to the rightward direction and negative equivalent to the leftward direction.

FIG. 41 illustrates the conditions following loading of the model shown in FIG. 35, showing horizontal principal stress using the scale of FIG. 40. Being so urged by 560-1, a region 522-1 of the simulated bone tissue 517-1, adjacent to the side of 510-1 that is predominantly facing the direction from which the simulated load 560-1 originates, is compressed against an adjacent portion of the modeled threads 511-1 and shaft 513-1.

Being so compressed, concentrations 524-1 of horizontal principal stress are shown with magnitudes in the simulated bone tissue portions 522-1 of negligible value close to zero. This region of bone is simultaneously loaded with vertical principal stress that at least reduces the risk of stress shielding.

Concurrent with the compression of the simulated bone tissue region 522-1 due to the urging of 517-1 by 560-1, simulated bone tissue in the region 523-1 of 517-1 that is positioned roughly mirrored to 522-1 across the implant axis 300-1 is shown to be exposed to horizontal stress concentrations 525-1 of 2.43 MPa, resulting mainly from the entrapment of bone tissue against 14-1 in the proximal threads. Any risk of exceeding physiological range in this region of bone tissue would be offset by the absence of high stress in the vertical component in this region.

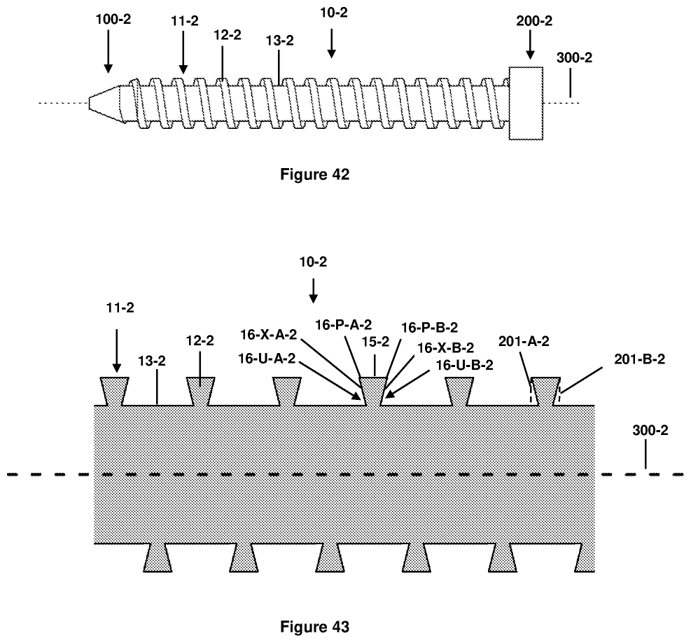

Referring to FIGS. 42 and 43, there is shown an embodiment of an orthopaedic implant device 10-2 according to the present invention., As shown in FIG. 43, a partial sectional view of the implant device of FIG. 42 is depicted where the plane of the section cut has a normal vector that is also normal to 300-2, and the portion shown is roughly near the midpoint between 100-2 and 200-2.

The thread profile 12-2 of each thread 11-2 possesses at least a distal undercut facet 16-X-A-2 provided by the leading edge that is a surface or curve that begins at the most distal portion of 12-2 and extends generally away from 300-2 and towards 100-2 until, a crest 15-2 that is generally flat or rounded and is also generally parallel to 300-2, and a proximal undercut 16-X-B-2 provided by the trailing edge that is a surface or curve that begins at the most proximal point of 15-2 and extends generally towards 300-2 or 100-2.

The portions of crest 15-2 and undercut facet 16-X-A-2 that are nearest to the distal end 100-2 of the implant 10-2 meet a connecting feature 16-P-A-2 which may be a point, edge, fillet, facet, chamfer or similar feature. The portions of crest 15-2 and undercut facet 16-X-B-2 that are nearest to the proximal end 200-2 of the implant 10-2 meet a connecting feature 16-P-B-2 which may be a point, edge, fillet, facet, chamfer or similar feature. By projecting a datum line 201-2 from the most distal portion of 16-P-A-2 towards 300-2 until reaching 13-2, an undercut void space 16-U-A-2 may be formed. By projecting a datum line 201-2 from the most proximal portion of 16-P-B-2 towards 300-2 until reaching 13-2, an undercut void space 16-U-B-2 may be formed. In cases where 10-2 is inserted into bone tissue, these undercut void spaces 16-U-A-2 and 16-U-B-2 may be occupied by a portion of bone tissue.

Figure illustrates an embodiment of the present invention, an orthopaedic implant 10-3 used for fixing fractured or fragmented bone so that fragmented or fractured bone may be reduced in their correct anatomical positions while osteosynthesis, or healing, takes place. It has a distal end 100-3 for inserting into bone tissue, and a proximal end 200-3 that is operated by a surgeon, and a central axis 300-3 that follows a proximal to distal direction. Implant 10-3 also has threads 11-3 with a square angled-undercut profile 12-3 that follows a helical path around central shaft 13-3. The implant 10-3 may be formed from a biocompatible and/or bioresorbable and corrosion-resistant metal alloy, preferably stainless steel, titanium or cobalt-chromium alloy; the implant 10-3 may also be formed from a biocompatible and/or bioresorbable rigid or semi-rigid polymer suitable for orthopaedic implants, such as polyether ether ketone (PEEK); the implant 10-3 may also be formed from a biocompatible and/or bioresorbable rigid or semi-rigid ceramic material suitable for orthopaedic implants, such as silica or hydroxyapatite-based ceramics.

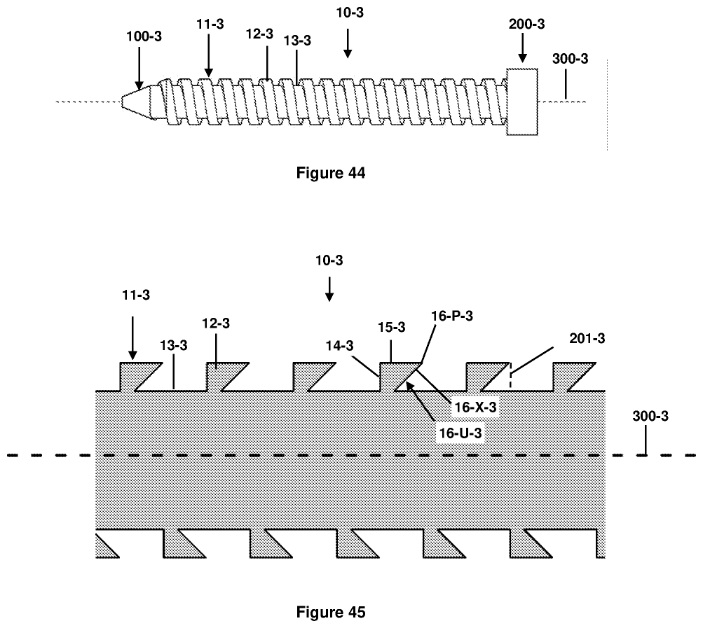

FIG. 45 shows an enlarged cross section of a further embodiment of an orthopaedic implant device according to the present invention as in FIG. 44, where the plane of the section cut has a normal vector that is also normal to 300-3, and the portion shown is roughly near the midpoint between 100-3 and 200-3.

The thread profile 12-3 of each thread 11-3 possesses at least a distal facet 14-3 provided by the leading edge, a crest 15-3 that is generally flat or rounded and is also generally parallel to 300-3, and an undercut 16-X-3 that is a surface or curve that begins at the most proximal point of 15-3 and extends generally towards 300-3 or 100-3.

The portions of crest 15-3 and undercut facet 16-X-3 provided by the trailing edge that are nearest to the proximal end 200-3 of the implant 10-3 meet a connecting feature 16-P-3 which may be a point, edge, fillet, facet, chamfer or similar feature. By projecting a datum line 201-3 from the most proximal portion of 16-P-3 towards 300-3 until reaching 13-3, an undercut void space 16-U-3 may be formed. In cases where 10-3 is inserted into bone tissue, this undercut void spaces 16-U-3 may be occupied by a portion of bone tissue.

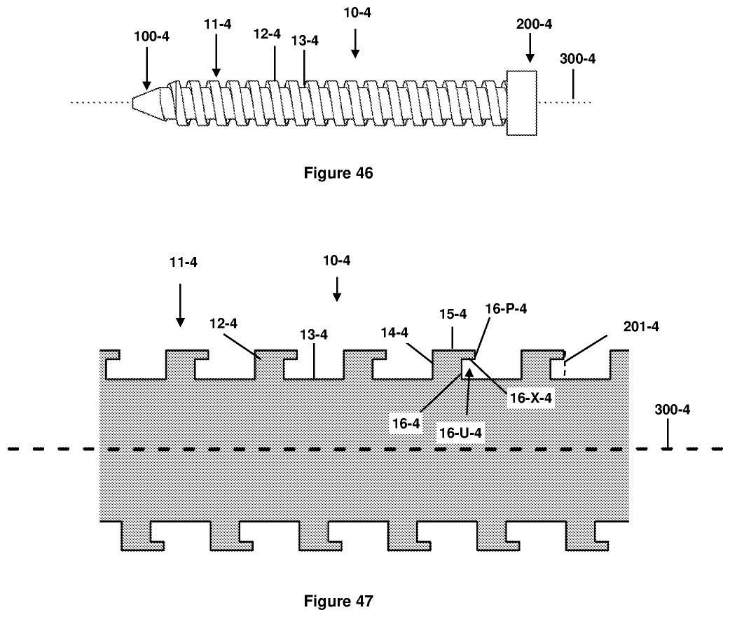

FIG. 46 shows another an embodiment of an orthopaedic implant device 10-4, according to the present invention, which is used for fixing fractured or fragmented bone so that fragmented or fractured bone may be reduced in their correct anatomical positions while osteosynthesis, or healing, takes place.

The implant device 10-4, has a distal end 100-4 for inserting into bone tissue, and a proximal end 200-4 that is operated by a surgeon, and a central axis 300-4 that follows a proximal to distal direction. Implant 10-4 also has threads 11-4 with a square angled-undercut profile 12-4 that follows a helical path around central shaft 13-4. The implant 10-4 may be formed from a biocompatible and/or bioresorbable and corrosion-resistant metal alloy, preferably stainless steel, titanium or cobalt-chromium alloy; the implant 10-4 may also be formed from a biocompatible and/or bioresorbable rigid or semi-rigid polymer suitable for orthopaedic implants, such as polyether ether ketone (PEEK); the implant 10-4 may also be formed from a biocompatible and/or bioresorbable rigid or semi-rigid ceramic material suitable for orthopaedic implants, such as silica or hydroxyapatite-based ceramics.

FIG. 47 shows the embodiment of FIG. 46, where the implant device 10-4 is shown here in section where the plane of the section cut has a normal vector that is also normal to 300-4, and the portion shown is roughly near the midpoint between 100-4 and 200-4.

The thread profile 12-4 of each thread 11-4 possesses at least a distal facet 14-4 provided by the leading edge, a crest 15-4 that is generally flat or rounded and is also generally parallel to 300-4, an undercut 16-X-4 that is a surface or curve that begins at the most proximal point of 15-4 and extends generally towards 300-4 or 100-4, and a proximal facet 16-4 that extends generally towards 300-4.

The portions of crest 15-4 and undercut facet 16-X-4 that are nearest to the proximal end 200-4 of the implant 10-4 meet a connecting feature 16-P-4 which may be a point, edge, fillet, facet, chamfer or similar feature. By projecting a datum line 201-4 from the most proximal portion of 16-P-4 towards 300-4 until reaching 13-4, an undercut void space 16-U-4 may be formed. In cases where 10-4 is inserted into bone tissue, this undercut void spaces 16-U-4 may be occupied by a portion of bone tissue.

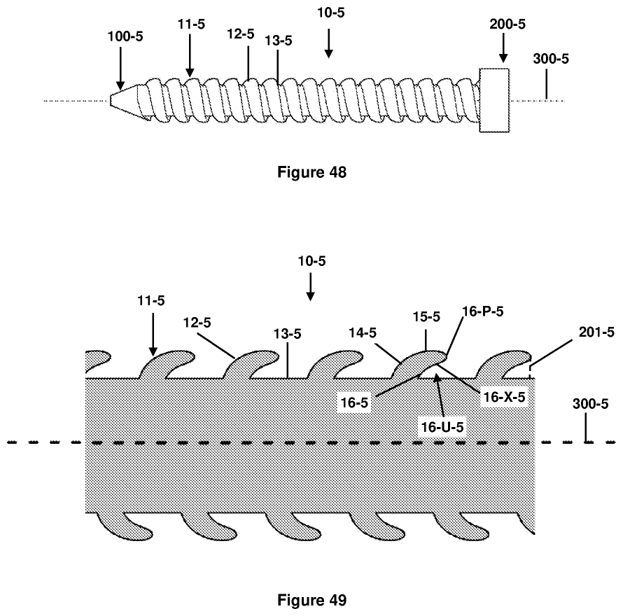

FIG. 48 shows yet a further embodiment of an orthopaedic implant 10-5 according to the present invention, used for fixing fractured or fragmented bone so that fragmented or fractured bone may be reduced in their correct anatomical positions while osteosynthesis, or healing, takes place.

It has a distal end 100-5 for inserting into bone tissue, and a proximal end 200-5 that is operated by a surgeon, and a central axis 300-5 that follows a proximal to distal direction. The implant device 10-5 also has threads 11-5 with a square angled-undercut profile 12-5 that follows a helical path around central shaft 13-5. The implant 10-5 may be formed from a biocompatible and/or bioresorbable and corrosion-resistant metal alloy, preferably stainless steel, titanium or cobalt-chromium alloy; the implant 10-5 may also be formed from a biocompatible and/or bioresorbable rigid or semi-rigid polymer suitable for orthopaedic implants, such as polyether ether ketone (PEEK); the implant 10-5 may also be formed from a biocompatible and/or bioresorbable rigid or semi-rigid ceramic material suitable for orthopaedic implants, such as silica or hydroxyapatite-based ceramics.

FIG. 49 shows the embodiment of FIG. 48, as shown here in section where the plane of the section cut has a normal vector that is also normal to 300-5, and the portion shown is roughly near the midpoint between 100-5 and 200-5. The thread profile 12-5 of each thread 11-5 possesses at least a distal facet or curve 14-5, a crest 15-5 that is generally flat or rounded and is also generally parallel to 300-5, an undercut 16-X-5 that is a surface or curve that begins at the most proximal point of 15-5 and extends generally towards 300-5 or 100-5, and a proximal facet 16-5 that extends generally towards 300-5.

The portions of crest 15-5 and undercut facet 16-X-5 that are nearest to the proximal end 200-5 of the implant 10-5 meet a connecting feature 16-P-5 which may be a point, edge, fillet, facet, chamfer or similar feature. By projecting a datum line 201-5 from the most proximal portion of 16-P-5 towards 300-5 until reaching 13-5, an undercut void space 16-U-5 may be formed. In cases where 10-5 is inserted into bone tissue, this undercut void spaces 16-U-5 may be occupied by a portion of bone tissue.

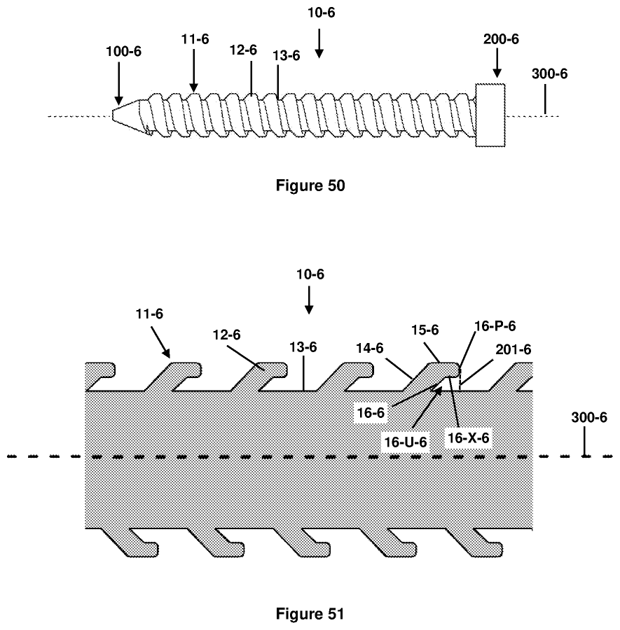

FIG. 50 shows yet another embodiment of an orthopaedic implant device 10-6 according to the present invention, whereby the orthopaedic implant device 10-6 is used for fixing fractured or fragmented bone so that fragmented or fractured bone may be reduced in their correct anatomical positions while osteosynthesis, or healing, takes place. It has a distal end 100-6 for inserting into bone tissue, and a proximal end 200-6 that is operated by a surgeon, and a central axis 300-6 that follows a proximal to distal direction. Implant 10-6 also has threads 11-6 with a square angled-undercut profile 12-6 that follows a helical path around central shaft 13-6. The implant 10-6 may be formed from a biocompatible and/or bioresorbable and corrosion-resistant metal alloy, preferably stainless steel, titanium or cobalt-chromium alloy; the implant 10-6 may also be formed from a biocompatible and/or bioresorbable rigid or semi-rigid polymer suitable for orthopaedic implants, such as polyether ether ketone (PEEK); the implant 10-6 may also be formed from a biocompatible and/or bioresorbable rigid or semi-rigid ceramic material suitable for orthopaedic implants, such as silica or hydroxyapatite-based ceramics.

FIG. 51 shows a sectional view of the orthopaedic implant device 10-6 of FIG. 50, shown here in section where the plane of the section cut has a normal vector that is also normal to 300-6, and the portion shown is roughly near the midpoint between 100-6 and 200-6. The thread profile 12-6 of each thread 11-6 possesses at least a distal facet or curve 14-6, a crest 15-6 that is generally flat or rounded and is also generally parallel to 300-6, an undercut 16-X-6 that is a surface or curve that begins at the most proximal point of 15-6 and extends generally towards 300-6 or 100-6, and a proximal facet 16-6 that extends generally towards 300-6.

The portions of crest 15-6 and undercut facet 16-X-6 that are nearest to the proximal end 200-6 of the implant 10-6 meet a connecting feature 16-P-6 which may be a point, edge, fillet, facet, chamfer or similar feature. By projecting a datum line 201-6 from the most proximal portion of 16-P-6 towards 300-6 until reaching 13-6, an undercut void space 16-U-6 may be formed. In cases where 10-6 is inserted into bone tissue, this undercut void spaces 16-U-6 may be occupied by a portion of bone tissue.

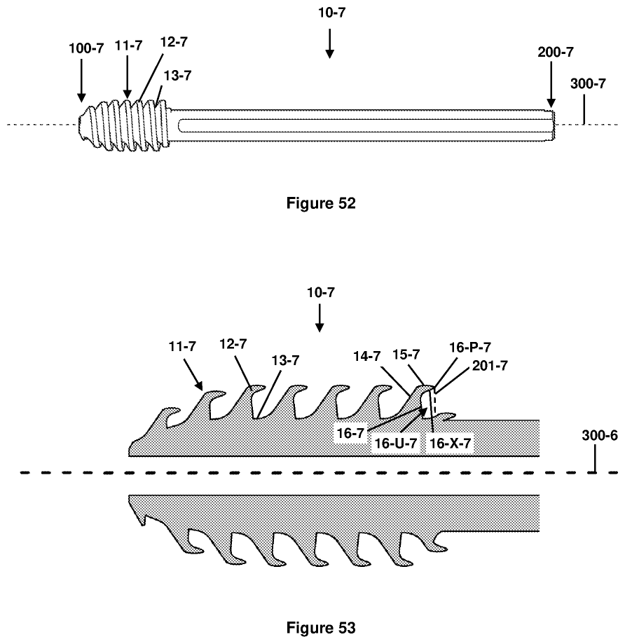

Referring to FIG. 52, there is shown still yet a further embodiment of an orthopaedic implant device 10-7 according to the present invention, used for fixing fractured or fragmented bone so that fragmented or fractured bone may be reduced in their correct anatomical positions while osteosynthesis, or healing, takes place. It has a distal end 100-7 for inserting into bone tissue, and a proximal end 200-7 that is operated by a surgeon, and a central axis 300-7 that follows a proximal to distal direction. Implant 10-7 also has threads 11-7 with a square angled-undercut profile 12-7 that follows a helical path around central shaft 13-7. The implant 10-7 may be formed from a biocompatible and/or bioresorbable and corrosion-resistant metal alloy, preferably stainless steel, titanium or cobalt-chromium alloy; the implant 10-7 may also be formed from a biocompatible and/or bioresorbable rigid or semi-rigid polymer suitable for orthopaedic implants, such as polyether ether ketone (PEEK); the implant 10-7 may also be formed from a biocompatible and/or bioresorbable rigid or semi-rigid ceramic material suitable for orthopaedic implants, such as silica or hydroxyapatite-based ceramics.

FIG. 53 shows an enlarged sectional view of a portion of the embodiment of FIG. 52an orthopaedic implant embodiment of the present invention as in FIG. 52, shown here in section where the plane of the section cut has a normal vector that is also normal to 300-7, and the portion shown is roughly near the midpoint between 100-7 and 200-7.

The thread profile 12-7 of each thread 11-7 possesses at least a distal facet or curve 14-7, a crest 15-7 that is generally flat or rounded and is also generally parallel to 300-7, an undercut 16-X-7 that is a surface or curve that begins at the most proximal point of 15-7 and extends generally towards 300-7 or 100-7, and a proximal facet 16-7 that extends generally towards 300-7. The portions of crest 15-7 and undercut facet 16-X-7 that are nearest to the proximal end 200-7 of the implant 10-7 meet a connecting feature 16-P-7 which may be a point, edge, fillet, facet, chamfer or similar feature. By projecting a datum line 201-7 from the most proximal portion of 16-P-7 towards 300-7 until reaching 13-7, an undercut void space 16-U-7 may be formed. In cases where 10-7 is inserted into bone tissue, this undercut void spaces 16-U-7 may be occupied by a portion of bone tissue.

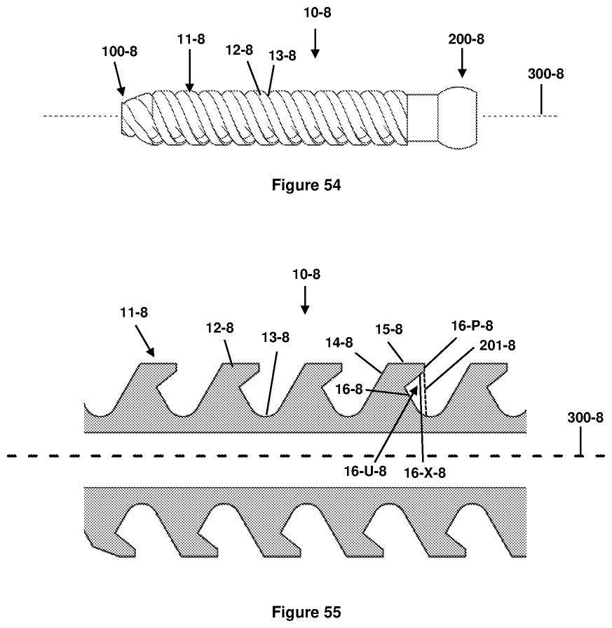

FIG. 54 shows still yet another embodiment of an orthopaedic implant device 10-8 according to the present invention, used for fixing fractured or fragmented bone so that fragmented or fractured bone may be reduced in their correct anatomical positions while osteosynthesis, or healing, takes place.

It has a distal end 100-8 for inserting into bone tissue, and a proximal end 200-8 that is operated by a surgeon, and a central axis 300-8 that follows a proximal to distal direction. Implant 10-8 also has threads 11-8 with a square angled-undercut profile 12-8 that follows a helical path around central shaft 13-8. The implant 10-8 may be formed from a biocompatible and/or bioresorbable and corrosion-resistant metal alloy, preferably stainless steel, titanium or cobalt-chromium alloy; the implant 10-8 may also be formed from a biocompatible and/or bioresorbable rigid or semi-rigid polymer suitable for orthopaedic implants, such as polyether ether ketone (PEEK); the implant 10-8 may also be formed from a biocompatible and/or bioresorbable rigid or semi-rigid ceramic material suitable for orthopaedic implants, such as silica or hydroxyapatite-based ceramics.

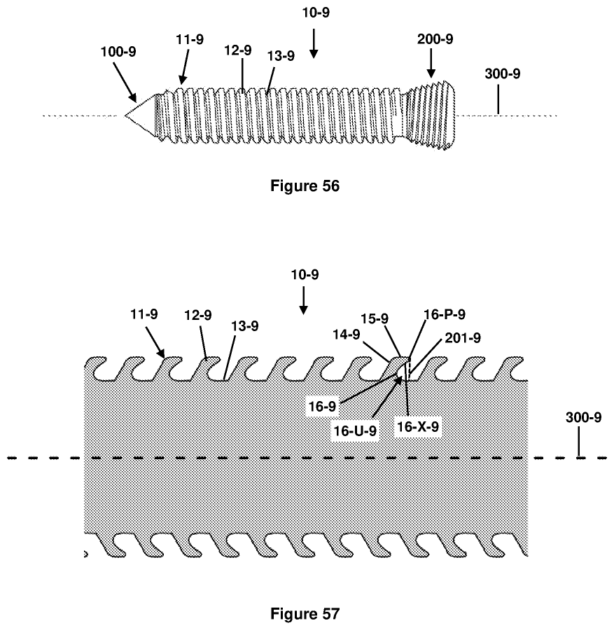

FIG. 55 shows an enlarged sectional view of the embodiment of FIG. 54i, shown here in section where the plane of the section cut has a normal vector that is also normal to 300-8, and the portion shown is roughly near the midpoint between 100-8 and 200-8. The thread profile 12-8 of each thread 11-8 possesses at least a distal facet or curve 14-8, a crest 15-8 that is generally flat or rounded and is also generally parallel to 300-8, an undercut 16-X-8 that is a surface or curve that begins at the most proximal point of 15-8 and extends generally towards 300-8 or 100-8, and a proximal facet 16-8 that extends generally towards 300-8. The portions of crest 15-8 and undercut facet 16-X-8 that are nearest to the proximal end 200-8 of the implant 10-8 meet a connecting feature 16-P-8 which may be a point, edge, fillet, facet, chamfer or similar feature. By projecting a datum line 201-8 from the most proximal portion of 16-P-8 towards 300-8 until reaching 13-8, an undercut void space 16-U-8 may be formed. In cases where 10-8 is inserted into bone tissue, this undercut void spaces 16-U-8 may be occupied by a portion of bone tissue.

FIG. 56 shows an alternate embodiment of an orthopaedic implant 10-9 according to the present invention, used for fixing fractured or fragmented bone so that fragmented or fractured bone may be reduced in their correct anatomical positions while osteosynthesis, or healing, takes place.

It has a distal end 100-9 for inserting into bone tissue, and a proximal end 200-9 that is operated by a surgeon, and a central axis 300-9 that follows a proximal to distal direction. Implant 10-9 also has threads 11-9 with a square angled-undercut profile 12-9 that follows a helical path around central shaft 13-9.

The implant 10-9 may be formed from a biocompatible and/or bioresorbable and corrosion-resistant metal alloy, preferably stainless steel, titanium or cobalt-chromium alloy; the implant 10-9 may also be formed from a biocompatible and/or bioresorbable rigid or semi-rigid polymer suitable for orthopaedic implants, such as polyether ether ketone (PEEK); the implant 10-9 may also be formed from a biocompatible and/or bioresorbable rigid or semi-rigid ceramic material suitable for orthopaedic implants, such as silica or hydroxyapatite-based ceramics.

FIG. 57 shows a sectional view of the embodiment of FIG. 56, shown here in section where the plane of the section cut has a normal vector that is also normal to 300-9, and the portion shown is roughly near the midpoint between 100-9 and 200-9. The thread profile 12-9 of each thread 11-9 possesses at least a distal facet or curve 14-9, a crest 15-9 that is generally flat or rounded and is also generally parallel to 300-9, an undercut 16-X-9 that is a surface or curve that begins at the most proximal point of 15-9 and extends generally towards 300-9 or 100-9, and a proximal facet 16-9 that extends generally towards 300-9.

The portions of crest 15-9 and undercut facet 16-X-9 that are nearest to the proximal end 200-9 of the implant 10-9 meet a connecting feature 16-P-9 which may be a point, edge, fillet, facet, chamfer or similar feature. By projecting a datum line 201-9 from the most proximal portion of 16-P-9 towards 300-9 until reaching 13-9, an undercut void space 16-U-9 may be formed. In cases where 10-9 is inserted into bone tissue, this undercut void spaces 16-U-9 may be occupied by a portion of bone tissue.

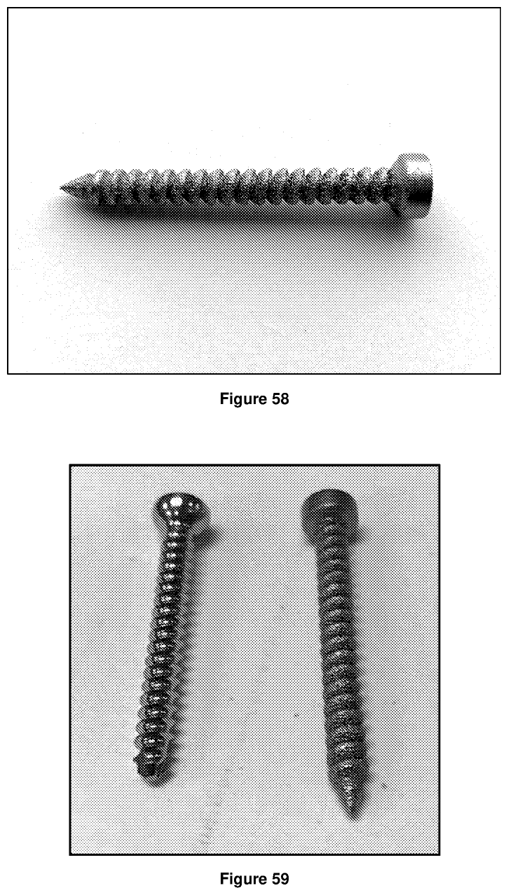

FIG. 58 is a photographic representation of a stainless steel three-dimensional printed prototype of an orthopaedic implant embodiment of the present invention following the design of 10-5 as presented in FIG. 48 and FIG. 49. The recent availability of this modern fabrication method allows for the production of orthopaedic screws with undercut features as are present in various embodiments of the present invention.

FIG. 59 is a photographic representation of, at left, a typical Prior Art AO-style bone screw used conventionally by those skilled in the art, and, at right, the prototype 10-5 as shown in FIG. 58. Both screws are of approximately the same major dimensions, being 40 mm in length and 4.4-4.5 mm in maximum diameter. Both screws are fabricated from stainless steel.

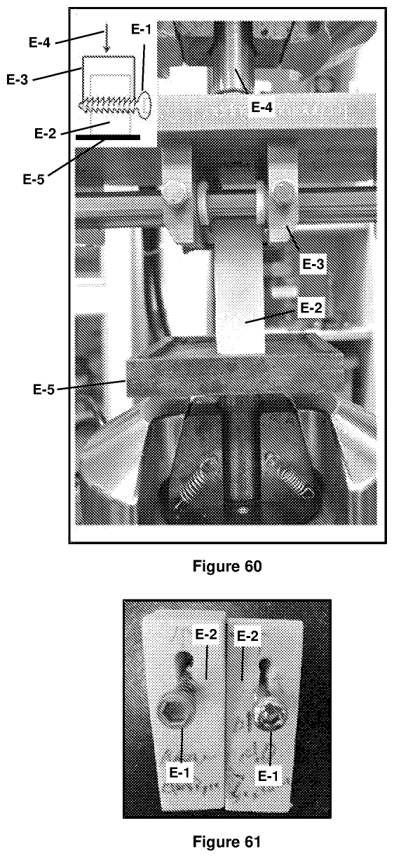

FIG. 60 is a diagram showing the experimental setup of a comparison between the two screws shown in FIG. 59. Each screw E-1 is inserted into its own separate block E-2 of 10 g/cc polyurethane foam (Sawbones ASTM Type 10) measuring 30×30×100 mm, pre-drilled with a 3 mm diameter pilot through-hole on one of the 30×100 mm sides, with the hole directed normal to the surface. Each E-1 is then pushed through is corresponding E-2 with at a displacement rate of 1 mm per minute via a force applied by a hydraulic press E-4 applied evenly on both distal and proximal ends simultaneously by a steel armature E-3. Force was measured by a loadcell E-5 below E-2, to a depth 8 mm.

FIG. 61 is a photographic representation showing the effect is the displacement experiment described in FIG. 60, on E-2.

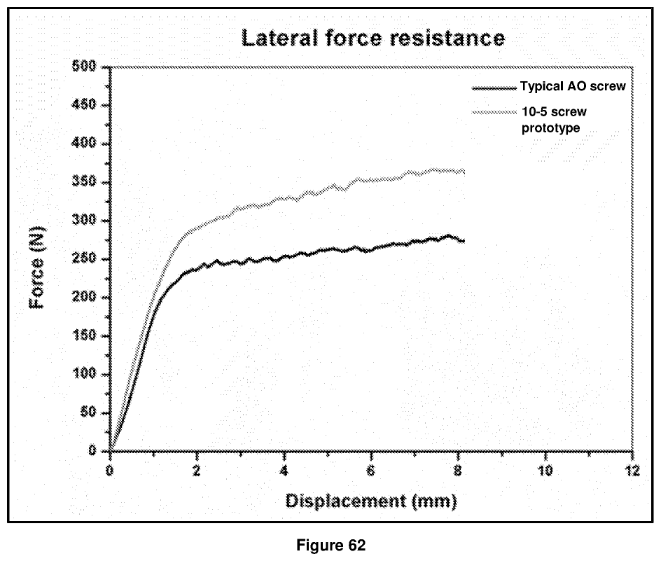

FIG. 62 is a graph of the force versus displacement result of the displacement experiment described in FIG. 60, showing empirical evidence of the utility of the present invention in terms of reducing orthopaedic implant migration and cut-out under loads perpendicular to the major axis of the implant.

As has been demonstrated and described above, the present invention provides an implant device which provides an improved load transfer, in particular lateral load transfer between the implant device and adjacent bone, by way of the novel thread portion of the implant device.

The present invention provides the dual advantages of (1) reducing excessive localised bone-damaging compressive stresses induced in bone material adjacent the thread portion of the implant device whilst providing a more uniform load transfer profile, and (2) inducing localised stress in bone material adjacent the thread portion of the implant device in regions whereby negligible load is imparted to such adjacent bone.

The advantages provided by the present invention include providing a localised stress environment which prevents or reduces localised trauma to bone tissue, and induced localised stresses to as to prevent or reduce bone resorption due to stress shielding.

Such a localised stress field assists in:
maintaining integrity of the bone/implant interface and stability of the bone/implant system,
reducing migration of the implant device through bone tissue,
reducing movement of the implant relative to adjacent bone tissue,
reducing bone loss through stress shielding and crushing and damage to bone adjacent the implant device, and
preventing aseptic loosening, which may precipitate major implant/system failure, or bone or implant failure.

As will be understood, the recess or undercut of the thread portion of the implant device as described above is exemplary, and numerous other thread profiles may be utilised in other or alternate embodiments of the invention.

Further, depending on the implant type, and different loading regime requirements, different thread portion geometries, sizes and shapes may be implemented on the implant accordingly.

The present invention is applicable to numerous types of implant devices and surgical technical fields.

Examples of some types of bone application screws to which the thread portion of the present invention may be incorporated with include:

1) Solid core, cannulated, and cannulated/fenestrated screws, nails, and anchors

2) Titanium, stainless steel, and polymer (both absorbable and non-absorbable)

3) Fully threaded, partially threaded, threaded/bladed

4) Non-self-tapping, self-tapping, self-drilling, self-drilling/self-tapping

5) Cortical, cancellous, pedicle, Herbert, malleolar, sliding screws, nails and anchors

6) Neutralization, lag, reduction, and position screws, nails, and anchors

Implant devices of the present invention may be deployed in various parts of anatomy, including arm, shoulder, forearm, wrist, hand, fingers; leg, hip, femoral shaft, knee, tibial shaft, fibial shaft, ankle, foot, toes; pelvis; spine; bones of the torso; neck; and maxillofacial, dental, and cranial applications.

Further, implant devices of the present invention are applicable to numerous surgical specialties including trauma, spine, extremities, sports, dental, maxillofacial, neurological specialties.

Whilst reference to the application of the implant device according to the present invention may generally be to a human subject, as will be understood the present invention may also be applicable to animals and veterinary applications,

1. An implant device for engagement with a bone of a subject, said implant device comprising a distal end, a proximal end, a central shaft extending therebetween and a longitudinal central axis;
said implant device further including a helical thread portion extending circumferentially about said central shaft and extending from the distal end towards the proximal end thereof, and a root at the base of the helical thread portion adjacent the central shaft, said helical thread portion including:
a leading edge and a trailing edge both extending at least radially outwardly from the central shaft and defining the thread portion therebetween, with the root of the thread portion defined therebetween in a direction of the longitudinal central axis of the implant device;
wherein said leading edge faces in a direction of at least towards the distal end of the implant device, and said trailing edge faces at least in a direction of towards the proximal end of the implant device; and
wherein a portion of the trailing edge extends in a direction towards the proximal end of the implant further than the most proximal portion of the root of the thread portion such that said portion of the trailing edge forms a recess between the central shaft and the trailing edge.

2. An implant device according to claim 1, wherein the portion of said trailing edge defining said recess provides for abutment and engagement with bone tissue of a subject disposed within said recess.

3. An implant device according to claim 1 or claim 2, wherein the thread portion further includes a crest portion at the crest of the thread portion.

4. An implant device according to claim 3, wherein the thread portion extends in at least a direction of from the distal end towards the proximal end, and wherein said crest portion forms a radially outward portion of the thread portion.

5. An implant device according to claim 4, wherein the crest portion provides an engagement surface for abutment and engagement with bone of a subject radially disposed from said thread portion.

6. An implant device according to claim 5, wherein said engagement surface of said crest portion, upon engagement with radially disposed bone adjacent the thread portion, provides for distribution of stress induced in said bone adjacent the crest portion along said engagement surface, and said engagement surface provides for reducing stress concentration in bone adjacent said crest portion.

7. An implant device according to claim 5 or claim 6, wherein the crest portion has a greater longitudinal length than that of the root portion in the direction of the longitudinal central axis of the implant device.

8. An implant device according to claim 5 or claim 6, wherein the longitudinal length of the thread portion from the most distal portion of the most proximal portion of the thread portion is greater than the length of the root of the thread portion.

9. An implant device according to any one of claims 5 to 8, wherein the leading edge of the thread portion includes a first facet for abutment and engagement with bone tissue of a subject, and wherein the trailing edge of thread portion includes a second facet for abutment and engagement with bone tissue of a subject, and wherein said crest portion is disposed between the first facet and the second facet.

10. An implant device according to claim 9, wherein the first facet has a substantially planar surface and extends substantially radially outwardly from the distal side of the root portion at the central shaft and extends towards the crest portion.

11. An implant device according to claim 9 or claim 10, wherein the second facet extends from the proximal side of the root portion at the central shaft and extends towards the crest portion.

12. An implant device according to claim 9 or claim 10, wherein the second facet is substantially planar and extends from the proximal side of the root portion at the central shaft and extends towards the crest portion at an inclination to the central shaft.

13. An implant device according to claim 9 or claim 10, wherein the trailing edge further includes a third facet, wherein the second and third facets have a substantially planar surface, and wherein the second facet and extends from the proximal side of the root portion at the central shaft and extends towards the third facet, and the third facet extends towards the crest portion.

14. An implant device according to claim 9 or claim 10, wherein the trailing edge further includes a third facet, wherein the second and third facets have a substantially planar surface, and wherein the second facet extends substantially radially outwardly from the proximal side of the root portion at the central shaft and extends towards the third facet, and the third facet extends in an inclined direction of from the second facet radially outwardly and proximally towards the crest portion.

15. An implant device according to claim 9 or claim 10, wherein the trailing edge further includes a third and a fourth facet, wherein the second and third and fourth facets have a substantially planar surface, and wherein the second facet extends substantially radially outwardly from the proximal side of the root portion at the central shaft and extends towards the third facet, and wherein the third facet extends in an direction substantially parallel to the shaft portion from the second facet and towards the fourth facet, and wherein the fourth facet extends from the third facet substantially radially outwardly from the third facet and towards the crest portion.

16. An implant device according to any one of claims 5 to 8, wherein the engagement surface of the crest portion is substantially planar and parallel to the longitudinal axis.

17. An implant device according to any one of claims 5 to 8, wherein the engagement surface of the crest portion is a curved surface.

18. An implant device according to any one of claim 5 to 8, 16 or 17, wherein the engagement portion of the crest portion is at least partially provided by the leading edge.

19. An implant device according to any one of claim 5 to 8, 16, 17 or 18, wherein the engagement portion of the crest portion is at least partially provided by the trailing edge.

20. An implant device according to any one of the preceding claims, wherein the recess is sized and shaped so as to reduce stress concentration induced in bone in respect of bone engaged with and adjacent the tread portion.

21. An implant device according to any one of the preceding claims, wherein the recess is sized and shaped such that upon the implant device and adjacent bone in which the device is embedded being urged towards each other on a first side of the implant, at least a portion of the trailing edge of the thread portion is urged against bone disposed within the recesses on the opposed side of the implant device.

22. An implant device according to any one of the preceding claims, wherein the thread portion has a constant cross-sectional area and geometry.

23. An implant device according to any one of claims 1 to 21, wherein the thread portion has a varying cross-sectional area and geometry.

24. An implant device according to any one of the preceding claims, wherein the thread portion has a constant thread pitch.

25. An implant device according to any one of claims 1 to 23, wherein the thread portion has a varying a constant thread pitch.

26. An implant device according to any one of the preceding claims, wherein the implant device is formed from a metal or metal alloy material.

27. An implant device according to claim 26, wherein the metal or metal alloy material is selected from the group including stainless steel, titanium, titanium alloy, cobalt-chromium alloy or the like.

28. An implant device according to any one of claims 1 to 25, wherein the implant device is formed from a polymeric material or polymer based material.

29. An implant device according to claim 28, wherein the polymeric material or polymer based material is polyether ether ketone (PEEK).

30. An implant device according to any one of the preceding claims, wherein the implant device is a bone screw.

31. An implant device according to any one of claims 1 to 29, wherein the implant device is an orthopaedic locking screw.

32. An implant device according to any one of claims 1 to 29, wherein the implant device is a pedicle screw device.

33. An implant device according to any one of claims 1 to 29, wherein the implant device is the femoral head engagement element of a dynamic hip screw.

34. An implant device according to any one of claims 1 to 29, wherein the implant device is bone suture anchor.

35. An implant device according to any one of claims to 29, wherein the implant device is an orthopaedic implant prosthesis device.

36. A kit comprising one or more implant devices according to any one of claims 1 to 29.

37. A kit according to claim 36, wherein the one or more implant devices is a bone screw.

38. A kit according to claim 36 or 37, further comprising one or more fracture fixation devices.

39. A system for fixing a first portion of bone relative to a second portion of bone, said system having 2 or more implant devices according to any one of claims 1 to 29 and a bridging member, wherein a first implant device is engageable with the first portion of bone and a second implant device is engageable with the second portion of bone, wherein the distal ends of the implant devices are engageable with said portions of bone and the proximal ends are engageable with said bridging member.

40. A system according to claim 39, wherein the one or more implant devices are pedicle screws and the bridging member is a rod, and the system is a spinal fusion system.

41. A system according to claim 40, wherein the rod is adjustable so as to provide adjustable movement of the first portion of bone and the second portion of bone relative to each other.

42. A system according to claim 39, wherein the system is a trauma fixation system.

43. An implant device for engagement with a bone of a subject, said implant device comprising a distal end, a proximal end, a central shaft extending therebetween and a longitudinal central axis;
said implant device further including a helical thread portion extending circumferentially about said central shaft and extending from the distal end towards the proximal end thereof, and a root at the base of the helical thread portion adjacent the central shaft, said helical thread portion including:
a leading edge and a trailing edge both extending at least radially outwardly from the central shaft and defining the thread portion therebetween, with the root of the thread portion defined therebetween in a direction of the longitudinal central axis of the implant device, and wherein said leading edge faces in a direction of at least towards the distal end of the implant device, and said trailing edge faces at least in a direction of towards the proximal end of the implant device;
a crest portion at the crest of the thread portion, wherein the thread portion extends in at least a direction of from the distal end towards the proximal end and provides a recess between the central shaft and the thread portion for abutment and engagement with bone adjacent the thread portion, and wherein said crest portion forms a radially outward portion of the thread portion and includes an engagement surface for abutment and engagement with bone of a subject radially disposed from said thread portion

44. An implant device according to claim 43, wherein said engagement surface of said crest portion, upon engagement with radially disposed bone adjacent the thread portion, the crest portion provides for distribution of stress induced in said bone adjacent the crest portion along said engagement surface, and said engagement surface provides for reducing stress concentration in bone adjacent said crest portion.

45. An implant device according to claim 43 or claim 44, wherein the recess is sized and shaped such that upon the implant device and adjacent bone in which the device is embedded being urged towards each other on a first side of the implant, at least a portion of the recess is urged against bone disposed within the recesses on the opposed side of the implant device.

46. An implant device according to any one of claims 43 to 45, wherein the trailing edge forms a recess between the central shaft and the trailing edge.

47. An implant device according to any one of claims 43 to 46, wherein the implant device is a bone screw.

48. An implant device according to any one of claims 43 to 46, wherein the implant device is an orthopaedic locking screw.

49. An implant device according to any one of claims 43 to 46, wherein the implant device is a pedicle screw device.

50. An implant device according to any one of claims 43 to 46, wherein the implant device is the femoral head engagement element of a dynamic hip screw. 10

51. An implant device according to any one of claims 43 to 46, wherein the implant device is bone suture anchor.

52. An implant device according to any one of claims 43 to 46, wherein the implant device is an orthopaedic implant prosthesis device.

53. A kit comprising one or more implant devices according to any one of claims 43 to 46.

54. A kit according to claim 53, wherein the one or more implant devices is a bone screw.

55. A kit according to claim 53 or claim 54, further comprising one or more fracture fixation devices.

56. A system for fixing a first portion of bone relative to a second portion of bone, said system having 2 or more implant devices according to any one of claims 43 to 46 and a bridging member, wherein a first implant device is engageable with the first portion of bone and a second implant device is engageable with the second portion of bone, wherein the distal ends of the implant devices are engageable with said portions of bone and the proximal ends are engageable with said bridging member.

57. A system according to claim 56, wherein the one or more implant devices are pedicle screws and the bridging member is a rod, and the system is a spinal fusion system.

58. A system according to claim 57, wherein the rod is adjustable so as to provide adjustable movement of the first portion of bone and the second portion of bone relative to each other.

59. A system according to claim 57, wherein the system is a trauma fixation system.
